(19) United States
(12) Patent Application Publication
(10) Pub. No.: US 2023/0096120 A1
(43) Pub. Date: Mar. 30, 2023

(54) UNIVERSAL SHOULDER PROSTHESIS SYSTEM AND TOOLS

(71) Applicant: Raphael S.F. Longobardi, LLC, Old Tappan, NJ (US)

(72) Inventor: Raphael S.F. Longobardi, Old Tappan, NJ (US)

(21) Appl. No.: 17/907,930

(22) PCT Filed: Mar. 2, 2021
(86) PCT No.: PCT/US2021/020492

§ 371 Date: Aug. 29, 2022

Related U.S. Application Data
(60) Provisional application No. 62/984,250, filed on Mar. 2, 2020.
(60) Provisional application No. 63/029,414, filed on May 23, 2020.

(51) Int. Cl. A61F 2/40; A61B 17/17

(52) U.S. Cl. CPC A61F 2/4081; A61B 17/1778; A61F 2002/30253

(57) ABSTRACT

A modular shoulder prosthesis system, in at least one embodiment, provides flexibility in shoulder replacements and ability to switch between a traditional anatomic Total Shoulder Replacement (ta-TSR) to a reverse Total Shoulder Replacement (r-TSR). Optionally, the system provides for a modular adaptation for the glenoid side in a TSR. The system includes a baseplate, a modular component, a humeral base and a modular humeral component. The baseplate includes a base with at least two attachment points extending in from opposed outer circumferential sides of the base. The modular component and the modular humeral component configured to cooperate with each other. The baseplate and the humeral base, or alternatively a second baseplate, are capable of attachment to different modular components to facilitate both ta-TSR and r-TSR with a change in the attached modular component.

### I. FIELD OF THE INVENTION

The invention relates to a modular shoulder prosthesis system and/or individual system components that provide for flexibility in shoulder replacements and allows for a more efficient switch for a patient between a traditional anatomic Total Shoulder Replacement (ta-TSR) to a reverse Total Shoulder Replacement (r-TSR). In at least one embodiment, the system also, or alternatively, provides for a modular adaptation for the glenoid side in a Total Shoulder Replacement (TSR). In at least one embodiment, the invention relates to the tool(s) for implanting, extracting, and/or exchanging components of the system in a patient.

### II. BACKGROUND OF THE INVENTION

TSRs have evolved over the last 70 years, with the greatest degree of its evolution occurring within the past 20 years. The understanding of the complexity of the shoulder has resulted in the ability to better treat the multiple conditions that afflict the shoulder. Glenohumeral arthritis ranges from simple to complex due to etiology and deformity. Post traumatic glenohumeral arthritis, along with the deformity of both the glenoid and humeral head present challenges for the shoulder arthroplasty surgeon. Similarly, the problem of rotator cuff deficiency and rotator cuff arthropathy has resulted in the development of treatment and prosthetic designs specific to address the loss of the main motors of the shoulder.

Currently, there are two types of TSR—traditional anatomic total shoulder replacement (ta-TSR) and reverse total shoulder replacement (r-TSR). Ta-TSR utilizes resurfacing of the humeral head and glenoid in the setting of an intact and functioning rotator cuff. Glenohumeral arthritis has been treated with ta-TSR, the current gold standard being the resurfacing of the humeral head with a stemmed or metaphyseal component along with a replacement of the humeral head articular portion with a Cobalt-Chromium (Co—Cr) implant. Modularity of the humeral components allows for appropriate sizing of the head in diameter and thickness to match the resected articular surface of the patient.

To revise from the ta-TSR to r-TSR often requires removal of the glenoid component and reconstruction of the glenoid bone stock. Ta-TSR utilize all polyethylene glenoid components which become the typical point of failure for the ta-TSR. R-TSR utilizes a porous in-growth metal design with locking screws for glenoid fixation. Modularity has always centered on being able to change the humeral components from ta-TSR to r-TSR, for example, a humeral head component to a socket configuration.

The resurfacing of the glenoid has also evolved over the past 70 years. Originally, polyethylene bonded to metal, also known as metal-backed glenoids, was cemented into the glenoid bone. These failed at the polyethylene-metal interface due to stresses and edge loading of the component. What evolved was the use of all polyethylene components. First, all polyethylene with a keel was used, followed by all polyethylene with multiple pegs.

There was a higher rate of failure for the cemented keeled components, so currently the gold standard for glenoid resurfacing in ta-TSR is a cemented pegged, all polyethylene component.

The most common cause of failure of the ta-TSR is due to glenoid loosening secondary to rotator cuff failure/tear. The resulting superior migration of the humeral head, with concomitant change in the center of rotation (C.O.R.) from rotator cuff failure produces edge loading of the glenoid component. This asymmetric mechanical loading results in rocking and loosening of the polyethylene prosthesis from the cement and bone of the glenoid.

R-TSR evolved from the specific abnormal mechanics of the rotator cuff deficient shoulder, as previously described. In the rotator deficient condition, the deltoid muscle becomes the predominant motor, but in an inefficient manner. The deltoid muscle contraction functions to result in “hinged abduction” of the humeral head/humeral shaft. The humeral head and greater tuberosity lever on the undersurface of the acromion and superior portion of the glenoid. Ta-TSR is contra-indicated in the setting of rotator cuff deficiency, due to the known catastrophic results to the glenoid component.

The development of the r-TSR addresses the rotator cuff deficient, painful arthritic shoulder. The design of r-TSR is to maximize deltoid fiber length to allow more efficient contraction and function of the deltoid in elevation of the arm. The prosthetic components are designed to change the C.O.R. to one that is more inferior and medial to the native joint.

The design of r-TSR has also evolved over the past 20 years. The original “Grammont” style sought to inferiorly displace the humerus to maximize deltoid fiber length; this resulted in inferior scapular notching, leading to failure. The current revised designs include a C.O.R. which is more lateral and inferior to the native C.O.R. The implant design is for an in-growth trabecular metal baseplate with locking screws to secure the component to the bony glenoid. The relatively minimally curved glenoid is replaced with a glenosphere: a solid Co—Cr semi-spherical to ¾ spherical surface that attaches to the in-growth metal base-plate. This is typically through a combination of a Morse taper fit and center screw fixation. The glenosphere is typically inserted at an inferiorly directed version angle, between 5-10 degrees. This allows for inferior offset of the humerus, elongation of the deltoid muscle fibers and a joint reactive force in line with prosthetic alignment.

R-TSR already have asymmetric, higher shear and higher loading of the glenoid component, called the glenosphere and baseplate construct. Despite these greater loads, in growth metal baseplates with locking screws are not the cause of failure, due to the excellent bone incorporation and stability.

The r-TSR has a different humeral component design as well. Where the ta-TSR has the Co—Cr humeral head, the r-TSR had the Co—Cr glenosphere attached to the glenoid. The humerus had a stemmed component but attached to the top is a polyethylene cup or humeral cup to articulate with the glenosphere. The modularity of components, specifically glenosphere sizing and humeral cup sizing, allow for multiple permutations to achieve the most successful and stable construct.

Current long-term studies on viability of r-TSR have revealed that the construct of an in-growth metal baseplate with locking screws has excellent long-term fixation without evidence of loosening, even in osteoporotic bone.

### III. SUMMARY OF THE INVENTION

In at least one embodiment, a modular system will allow the surgeon to achieve either exchanges, humeral or glenoid component, without an extravagant amount of equipment to be used, or more complex operative procedures to be performed. A truly versatile and modular system would allow for a baseplate to accept either a traditional anatomic glenoid component or a reverse total shoulder glenosphere, without compromising long term security and function. At least one embodiment according to the invention will allow for all of this. As discussed below, tools that may be used to implant the described modular system(s) and allow for interchange of the modular components, and in further embodiments the removal of the baseplate if necessary.

The tools, in at least one embodiment, allow for a single glenoid component that can be used for traditional, anatomic TSR, primary reverse TSR and revision of anatomic to reverse TSR.

Different embodiments of the invention are directed at different tools for use in implanting, exchanging, and/or removing/extracting components that are part of the system. The tools include a drill guide, two different reamers, a baseplate inserter and/or extractor, and a humeral cutting guide with or without arms. Based on this disclosure, a person of ordinary skill in the art will appreciate that one or more of these tools may be useful with other shoulder prosthesis systems than those discussed in this disclosure.

In at least one embodiment, the baseplate will include a pair of notches extending up from a bottom, peripheral edge of the baseplate, which in at least one tool embodiment will provide an attachment point for implanting the baseplate onto the glenoid or humerus of the patient. In a further embodiment, these notches will provide an attachment point for a tool to remove the implanted baseplate, if necessary. In an alternative embodiment, there are a pair of slots open to the external periphery of the baseplate to provide an attachment point. In either embodiment, the slots or notches are located on opposed external peripheral surfaces of the baseplate. In a further embodiment, the number of slots or notches can be greater than two, while in a still further embodiment the slots/notches are evenly spaced around the exterior peripheral surface of the baseplate. The notches and slots are examples of attachment points. In at least one embodiment, the attachment points are aligned with notches providing a leverage point for removal of the modular component from the baseplate, while in other embodiments the attachment points and leverage points are not aligned with each other.

A modular shoulder prosthesis system including a baseplate having a base with a plurality of attachment holes passing therethrough, which may be omitted entirely or one centrally located attachment hole may be present, and at least two notches and/or slots on opposed external sides of the base, and a central stem extending from the base and axially centered with one of the plurality of attachment holes; a modular component (for the glenoid side) configured to be removably attached to the base, the modular component having a plug for insertion into at least one attachment hole of the base; a humeral base (a humeral stem or a second baseplate) having a receiving cavity extending in from one face; and a modular humeral component configured to cooperate with the modular component, the modular humeral component having a post configured for removable insertion into the receiving cavity of the modular humeral component, and wherein the baseplate is capable of attachment to different modular components and the humeral base is capable of attachment to different modular humeral components to facilitate both traditional anatomic total shoulder replacement and reverse total shoulder replacement with a change in the modular component and change in the modular humeral component. The ability of the baseplate to be used for either glenoid or humeral fixation to bone allows it to be a universal baseplate. In a further embodiment, the pair of notches extend down from a mounting surface of the base. In a further embodiment to either embodiment, the notches are located on the anterior and posterior sides of the base.

In a further embodiment to the previous embodiments the modular component includes a pair of protrusions extending from opposing sides of the modular component, the protrusions configured to align with the notches when the modular component is attached to the base. In a further embodiment to the embodiment of the prior paragraph, the modular component includes a pair of protrusions extending down from opposing sides of the modular component, the protrusions configured to engage with an interference fit the notches when the modular component is attached to the base. In a further embodiment to the above embodiments, the modular component includes a flange extending down from the outer circumferential edge such that the flange fits over and/or around the baseplate. In such an embodiment with the protrusions, the pair of protrusions are present on the interior peripheral sides of the flange. In a further embodiment to any of the previous embodiments, the plug of the modular component and/or the post of the modular humeral component include a Morse taper.

In a further embodiment to any of the previous embodiments, the modular component for a ta-TSR includes a base having a concave surface, and the plug extends from a surface opposite of the concave surface to be inserted into the glenoid. Further to the previous embodiment, the modular component includes Co—Cr. Further to the embodiments of the previous two paragraphs, the modular component for a r-TSR includes a base, a glenosphere extending from the base, and the plug extends from a surface of the base opposite the glenosphere to be inserted into the glenoid. Further to the previous embodiment, the glenosphere is approximately a three-quarters sphere or a hemi-spherical dome. Further to the previous two embodiments, the glenosphere includes Cobalt-Chromium. In at least one embodiment, the glenosphere includes an inferior tilt of approximately 10-degrees from a vertical axis perpendicular to the base, which creates an oblong shape of the glenosphere body, rather than a sphere or hemisphere seen typically in commercially available prosthetic.

In a further embodiment to any of the previous embodiments, the modular humeral component for a ta-TSR includes a base having an inner head; an outer shell over the inner head; and the post extending from the base on a surface opposing the inner head. Further to the previous embodiment, the inner head is a hemi-spherical dome and the outer shell is a hemi-spherical cap that fits over the inner head. Further to the previous two embodiments, the inner head includes Co—Cr and the outer shell includes a high-density polyethylene. In a further embodiment, there is dual-mobility between the outer shell and the concave surface of the modular component attached to the glenoid. Further to the embodiments of the previous three paragraphs, the modular humeral component for a r-TSR includes a base having a receiving cavity, and the post extends from a surface of the base opposing the receiving cavity to be inserted into the humeral base. Further to the previous embodiment, the base includes high-density polyethylene to form a concave surface in the receiving cavity or the modular humeral component further includes a shell inserted into the receiving cavity of the base. Further to the previous shell embodiment, the base includes Co—Cr and the shell includes a high-density polyethylene. The embodiments of this paragraph and the previous paragraph are used in combination where one component has a glenosphere and the other component has a concave surface.

In a further embodiment to any of the previous embodiments, the system further including a plurality of attachment mechanisms, wherein the attachment holes of the base of the baseplate are configured to engage with the attachment mechanisms. Further to the previous embodiment, the attachment mechanisms include variable angle locking screws.

Further to the embodiments of the previous three paragraphs, the modular component includes a passageway through which a fastener passes to engage the respective glenoid base. Further to the embodiments of the last two paragraphs, the modular humeral component has an eccentrically placed plug.

A modular shoulder prosthesis system including a baseplate having a base with a plurality of attachment holes passing therethrough, which may be omitted entirely or one centrally located attachment hole may be present, and at least two notches and/or slots on opposed external peripheral sides of the base, and a central stem extending from the base and axially centered having a receiving cavity; a modular component configured to be removably attached to the base, the modular component having a plug for insertion into the receiving cavity of the base, and wherein the baseplate is capable of attachment to different modular components to facilitate both traditional anatomic total shoulder replacement and reverse total shoulder replacement with a change in the modular component. Further to the previous embodiment, the pair of notches may extend down from a mounting surface and along the sides of the base. Further to the embodiments of this paragraph, the modular component includes a pair of protrusions extending from (including down from) opposing sides of the modular component, the protrusions configured to overlap with the notches when the modular component is attached to the base and/or to engage with an interference fit the notches when the modular component is attached to the base. In a further embodiment to the above embodiments, the modular component includes a flange extending down from the outer circumferential edge such that the flange fits over and/or around the baseplate. In such an embodiment with the protrusions, the pair of protrusions are present on the interior peripheral sides of the flange.

Further to the embodiments of the previous paragraph, the plug of the modular component includes a Morse taper and/or threaded surface for engagement of the baseplate. In an alternative embodiment, the component including the plug have a passageway passing therethrough for a fastener to engage the component and/or any attachment mechanism.

Further to the embodiments of the previous two paragraphs, the modular component for a ta-TSR includes a base having a concave surface, and the plug extends from a surface opposite of the concave surface. Further to the previous embodiment, the modular component includes Co—Cr. Further to the embodiments of the previous two paragraphs, the modular component for r-TSR includes a base, a glenosphere extending from the base, and the plug extends from a surface of the base opposite the glenosphere. Further to the previous embodiment, the glenosphere is approximately a three-quarters sphere. Further to the previous two embodiments, the glenosphere includes Co—Cr.

Further to the embodiments of the previous three paragraphs, the system further including at least one attachment mechanism, wherein the attachment holes of the base of the baseplate are configured to engage with the attachment mechanism. Further to the previous embodiment, the attachment mechanisms include variable angle locking screws.

Further to the previous embodiments and in a further embodiment, when the humeral stem is the second baseplate configured for attachment to the humerus, the second baseplate is larger than the glenoid baseplate.

In at least one embodiment, a modular shoulder prosthesis system including: a first baseplate configured to attach to a glenoid, the first baseplate having a base with a plurality of attachment holes passing therethrough, which may be omitted, and at least two notches and/or slots on opposed external peripheral sides of the base, the notches in at least one embodiment extend down from a mounting surface of the base, and a central stem extending from the base and axially centered with one of the plurality of attachment holes; a modular component configured to be removably attached to the base, the modular component having a plug for insertion into at least one attachment hole of the base; a second baseplate configured to attach to a humerus, the second baseplate having a receiving cavity extending in from one face; and a modular humeral component configured to cooperate with the modular component, the modular humeral component having a post configured for removable insertion into the receiving cavity of the second baseplate, and wherein the first baseplate is capable of attachment to different modular components and the second baseplate is capable of attachment to different modular humeral components to facilitate both traditional anatomic total shoulder replacement and reverse total shoulder replacement with a change in the modular component and change in the modular humeral component. In a further embodiment, the variously described modular components and modular humeral components of the summary section may be used in this system.

Further to the previous embodiments, each glenoid baseplate or humeral base depending on the embodiment includes three or more notches facing externally outward, the modular component and the modular humeral component are each configured to have the two flanges attach to two notches of the respective baseplate or humeral base when implanted to provide a selectable orientation relative to the other of the modular component and the modular humeral component.

Further to the above embodiments, the glenoid baseplate is used on the humerus side although in a further embodiment it is resized.

### IV. BRIEF DESCRIPTION OF THE DRAWINGS

Any cross-hatching present in the figures is not intended to identify or limit the type of material present for the element shown in cross-section. In figures that include multiple elements shown in cross-section, the cross-hatching will be different directions for the different elements.

FIGS. 1A-1C illustrate a baseplate. FIG. 1A illustrates a top view. FIG. 1B illustrates a side view with phantom lines illustrating the internal construction. FIG. 1C illustrates an alternative cross-section of the baseplate for an alternative attachment with a glenosphere modular component from a view from superiorly of the glenosphere. FIGS. 1D and 1E illustrate side views of alternative baseplates with phantom lines illustrating the internal construction according to at least two embodiments of the invention. FIG. 1F illustrates a side view with phantom lines illustrating the internal construction according to at least one embodiment of the invention. FIGS. 1G-1J illustrate an alternative baseplate. FIGS. 1G and 1H illustrate perspective views from the top and bottom, respectively. FIG. 1I illustrates a side view. FIG. 1J illustrates a cross-section taken through a diameter through the center of the notches.

FIGS. 2A and 2B illustrate a modular glenoid component. FIG. 2B is a cross-section taken at 2B, 2F-2B, 2F in FIG. 2A, which illustrates a top view of the modular glenoid component. FIG. 2C illustrates a top view of an alternative modular glenoid component. FIG. 2D illustrates a side view of another alternative modular glenoid component. FIG. 2E illustrates a bottom view of another alternative modular glenoid component according to at least one embodiment of the invention. FIG. 2F is a cross-section taken at 2B, 2F-2B, 2F in FIG. 2A that illustrates an alternative modular glenoid component according to at least one embodiment of the invention. FIG. 2G illustrates a cross-section of a modular glenoid component with a flange engaging a baseplate according to at least one embodiment of the invention. FIG. 2H illustrates a cross-section of a modular glenoid component with a flange and a pair of protrusions engaging a baseplate according to at least one embodiment of the invention. The cross-section is taken along a diameter. FIGS. 2H and 2L are similar figures with FIG. 2H being a purely “2D” representation with a cross-section, whereas FIG. 2L is a “3D” representation with a cross-section of a modular glenoid component engaged with a baseplate. FIGS. 2I-2L illustrate an alternative glenoid component depicting top, perspective, bottom, and cross-section views, respectively.

FIGS. 3A-3D illustrate glenosphere components. FIG. 3A illustrates a side view. FIG. 3B is a cross-section taken along a vertical plane taken at 3B, 3C, 3H-3B, 3C, 3H in FIG. 3D, which illustrates a face-on view or view from lateral shoulder view. FIG. 3C illustrates an alternative glenosphere component as a cross-section taken at 3B, 3C, 3H-3B, 3C, 3H in FIG. 3D. FIG. 3E illustrates a face-on view or view from lateral shoulder view of an alternative glenosphere component. FIG. 3F illustrates a top view of another alternative glenosphere component. FIG. 3G illustrates a cross-section of a glenosphere engaging a baseplate where the cross-section is taken along a diameter. FIG. 3H illustrates an alternative glenosphere component as a cross-section taken at 3B, 3C, 3H-3B, 3C, 3H in FIG. 3D. FIGS. 3I-3N illustrate an alternative glenosphere component including perspective and cross-section views with and without being attached to a baseplate. FIGS. 3I and 3J illustrate perspective views, FIG. 3K illustrates a bottom view, and FIGS. 3L and 3M illustrate cross-sections taken at respective diameters in FIG. 3N, which illustrates a top view. FIGS. 3L and 5B illustrate similar cross-sections for the respective illustrated glenospheres providing examples of inferior tilt shapes.

FIG. 4A illustrates a side view of a humeral head. FIG. 4B illustrates a cross-section of the humeral head with dual mobility head/cap/shell illustrated in FIG. 4A taken along a vertical plane taken through a diameter of the humeral head. FIGS. 4C-4I illustrate a humeral component with FIGS. 4H and 4I illustrating the humeral component, with dual mobility head/cap/shell, and with a baseplate.

FIGS. 5A and 5B illustrate cross-sections of examples of a modular glenoid component and humeral head implanted in bone.

FIGS. 6A-6C illustrate an alternative humeral cup. FIG. 6A illustrates a top view. FIG. 6B illustrates a cross-section taken at 6B-6B in FIG. 6A. FIG. 6C illustrates a side view of an alternative humeral cup. FIGS. 6D and 6E illustrate another alternative humeral cup. FIG. 6D illustrates a top view. FIG. 6E illustrates a cross-section taken at 6E-6E in FIG. 6D.

FIGS. 7A-7C illustrate a humeral cup shell. FIG. 7A illustrates a side view. FIG. 7B illustrates a cross-section taken at 7B-7B in FIG. 7C. FIG. 7C illustrates a bottom view. FIGS. 7D-7F illustrate a humeral cup shell. FIG. 7D illustrates a side view. FIG. 7E illustrates a cross-section taken at 7E-7E in FIG. 7F. FIG. 7F illustrates a bottom view.

FIGS. 8A and 8B illustrate cross-sections of examples of a baseplate being used in place of a humeral stem. FIG. 8B illustrates a 3-part assembly having similarities to FIG. 6E.

FIGS. 9A and 9B illustrate a cutting guide for use in implanting at least one shoulder prosthesis system discussed in this disclosure.

FIGS. 10A and 10B illustrate a reamer for use in implanting at least one shoulder prosthesis system discussed in this disclosure.

FIG. 11 illustrates a stem drill bit for use in implanting at least one shoulder prosthesis system discussed in this disclosure.

FIGS. 12A and 12B illustrate a baseplate inserter for use in implanting at least one baseplate discussed in this disclosure and in at least one embodiment is configured to remove an implanted modular component.

FIGS. 13A and 13B illustrate a baseplate extractor for use in removing an implanted baseplate.

FIGS. 14A and 14B illustrate a humerus cutting guide for use in implanting at least one system embodiment of the invention. The dashed lines are indicative of internal passageways and/or cavity. The presence of particular degree numbers is for illustration purposes and should not be deemed to limit the invention as it relates to the humerus cutting guide.

### V. DETAILED DESCRIPTION OF THE DRAWINGS

The invention in at least one embodiment includes tools for implanting, exchanging, and/or extracting one or more components of a modular shoulder replacement system having a glenoid baseplate configured for attachment to a patient's glenoid with at least one attachment mechanism, a humeral base such as a humeral stem or baseplate, and at least two modular components for attachment to the baseplate and/or humeral base. Examples of attachment mechanisms include screws, variable angle locking screws, and an impactable central cylinder. In at least one embodiment, the modular component includes an outer peripheral flange that overlaps the baseplate or the humeral base that in at least one embodiment include a pair of attachment points extending in from opposed external peripheral sides to engage notches in the baseplate or the humeral base.

The baseplate has a mounting surface for engagement of a modular glenoid component for a ta-TSR or a modular glenosphere component for a r-TSR. The mounting surface refers to the substantially planar surface of the baseplate opposite the glenoid (or humerus). Both the modular glenoid component and the modular glenosphere component are examples of a modular component. The attachment in at least one embodiment between the modular component and the baseplate is through, for example, a Morse taper, which may be a dual threaded Morse taper that is axially located with reference to the baseplate. In an alternative embodiment with or without the Morse taper, a torque limiting fastener, such as a screw or a bolt, is used to further secure the modular glenoid component to the baseplate by engaging the baseplate and/or the central attachment mechanism anchored in the patient's glenoid. In a further embodiment, the attachment is facilitated with a threaded connection where the modular component is screwed into the baseplate.

In at least one embodiment the modular shoulder replacement system further includes a humeral stem for attachment to a patient's humeral bone. The humeral stem having a receiving socket for insertion of a post (or other connection piece) from a modular humeral head for a ta-TSR or a modular humeral cup for a r-TSR. Both the modular humeral head and the modular humeral cup are examples of a modular humeral component. In at least one embodiment, the modular humeral component is attached to the humeral stem using, for example, a Morse taper, which may be a dual threaded Morse taper. In an alternative embodiment, the system includes a mounting base that is attached to the humeral stem component on to which the modular humeral head or modular humeral cup is attached. In an alternative embodiment with or without the Morse taper, a torque limiting fastener, such as a screw or a bolt, is used to further secure the modular humeral component to the humeral stem by engaging the receiving socket. In a further embodiment, the attachment is facilitated with a threaded connection where the modular humeral component is screwed into the humeral stem.

In an alternative embodiment, a baseplate used for the glenoid is adapted for use instead with the humerus in place of the humeral stem. In at least one embodiment, the glenoid baseplate (e.g., a first baseplate) and the humeral baseplate (e.g., a second baseplate) are identical. In at least one alternative embodiment, the humeral baseplate is larger than the glenoid baseplate. In a further embodiment, the humeral baseplate has a larger diameter (e.g., for the base and/or the stem), height, and/or thickness than the glenoid baseplate. Examples of diameters for the baseplates include about 25 mm, 27 mm, 29 mm, 32 mm, 33 mm, 37 mm, 41 mm, 42 mm, a range between 25 mm and 41 mm, a range of 25 mm to 33 mm, a range of 25 mm to 30 mm or a range of 25 mm to 27 mm where the measurements in this disclosure also include approximations given manufacturing tolerances and the ranges in a further embodiment include their respective endpoints. In at least one embodiment, the glenoid baseplate has a diameter of 27 mm while the humeral baseplate has a larger diameter although the same size diameter may be used on both the glenoid and humeral sides. Examples of baseplate sizes include 39 mm, 41 mm, 43 mm, 46 mm, 49 mm, 52 mm, and 55 mm, and in at least one further embodiment, these sizes are used for a humeral baseplate. Examples of humeral head thicknesses include 14 mm, 17 mm, and 20 mm. In at least one embodiment, the humeral head has an offset center in which there is a neutral position.

The baseplate and the humeral stem (or a second baseplate) are designed to remain in place while switching the modular component and the modular humeral component, respectively, to switch from a ta-TSR to a r-TSR. However, there may be times the baseplate needs to be extracted using one or more tools that engage the plurality of attachment points.

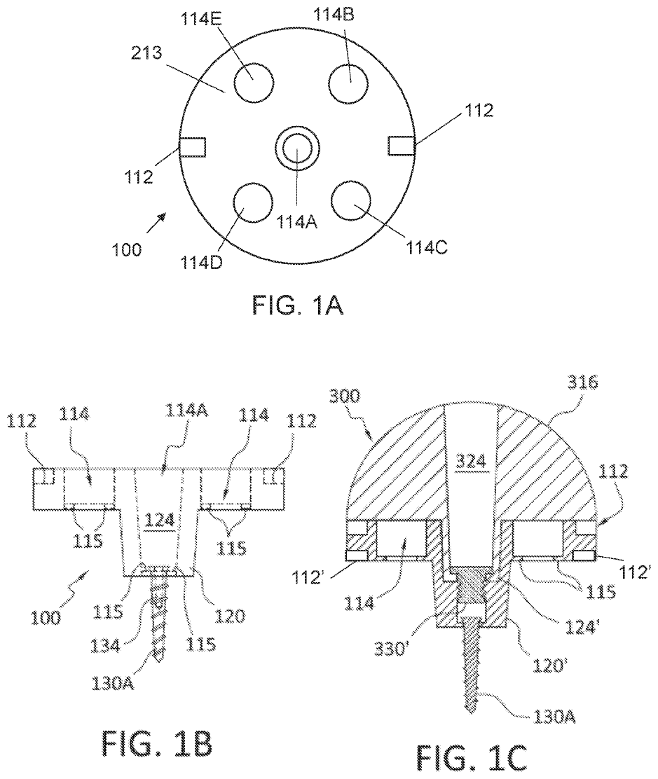

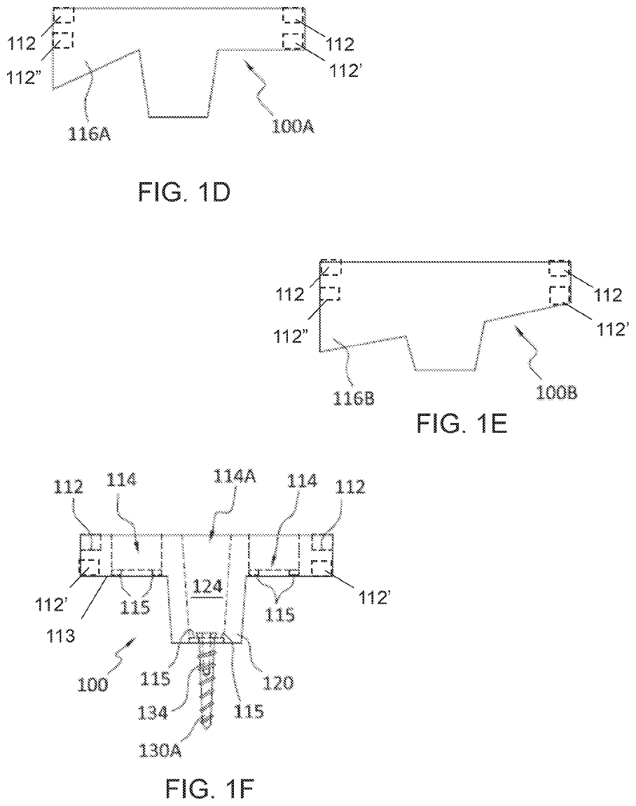

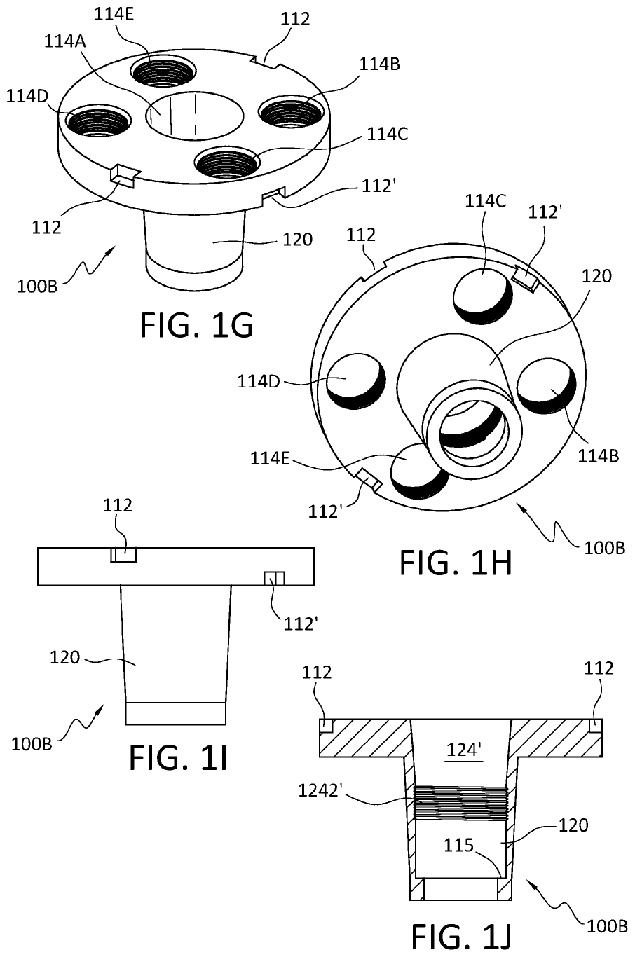

FIGS. 1A and 1B illustrate a baseplate 100. The illustrated baseplate 100 includes a base 110 and a stem 120 extending from the base 110. Although the base 110 is illustrated as being circular, the base 110 may be elliptical, oval, or other suitable shapes; in such an embodiment, the modular component may be shaped to match. Examples of the thickness of the base 110 include between 5 mm and 15 mm (with or without the end points), 5 mm, 7 mm, 10 mm, 12.5 mm, and 15 mm. In at least one embodiment, the baseplate 100 is made from an ingrowth trabecular metal over a metal core of, for example, steel, titanium or a combination of the two. The ingrowth trabecular metal facilitates bone ingrowth into the baseplate 100, for example to increase the strength of the connection between the bone and the baseplate 100 and the respective interface shear strength over time.

Although the stem 120 and 120′ in FIGS. 1B and 1C, respectively, are illustrated as a cylinder with a slight taper, in at least one embodiment, the stem 120 has tapered sides to match the modular component plugs. In a further embodiment, the stem 120 is substantially cylindrical.

The base 110 includes a mounting surface 111 on a side opposite of the side 113 from which the stem 120 extends. In at least one embodiment, the mounting surface 111 is substantially planar. The plane defined by the mounting surface 111 is approximately parallel to the glenoid resection plane after implantation. In an alternative embodiment, the plane defined by the mounting surface 111 is at an angle to the glenoid resection plane after implantation as illustrated, for example, in FIGS. 1D and 1E. The mounting surface 111 in at least one embodiment will be a sufficient height above the glenoid resection plane to allow access to notches 112, 112 or attachment points 112′, 112″.

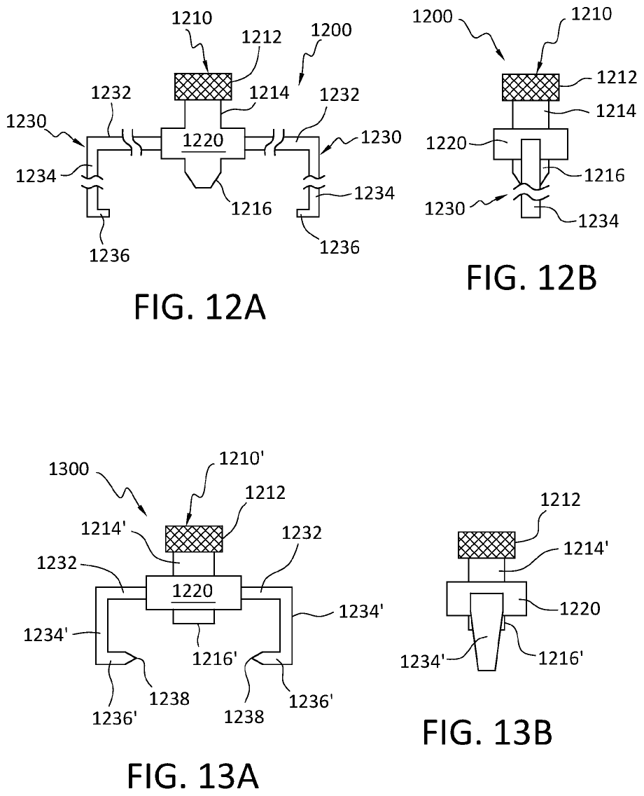

The illustrated base 110 includes a pair of attachment points, which are illustrated as notches 112′, 112′ in FIG. 1F and slot 112″ in FIGS. 1D and 1E. The notches extend up from outer circumferential sides of the bottom surface 113 from which the stem 120 extends. The notches 112′ and the slots 112″ are present on the outer circumferential sides of the base 110. Both types of attachment points are accessible from the exterior of the baseplate 100. The attachment points have sufficient width and depth to engage with an implanting/extracting instrument. In at least one further embodiment, the attachment points are configured to match the shape of the instrument used for implanting and/or extracting the baseplate 100 including the cross-section and/or depth of a finger of the instrument like the instruments illustrated in FIGS. 12A-13B. The instrument fingers are inserted into the attachment points 112/112′/112″ to assist with the implantation of the baseplate 100 onto the glenoid/humerus and, if necessary, is used to extract the baseplate 100 from the glenoid/humerus. Although two attachment points are discussed, additional attachment points could be added to the base 110. FIGS. 1D and 1E illustrate examples of how both a notch 112′ and a slot 112″ may be present to provide attachment points when a wedge is present.

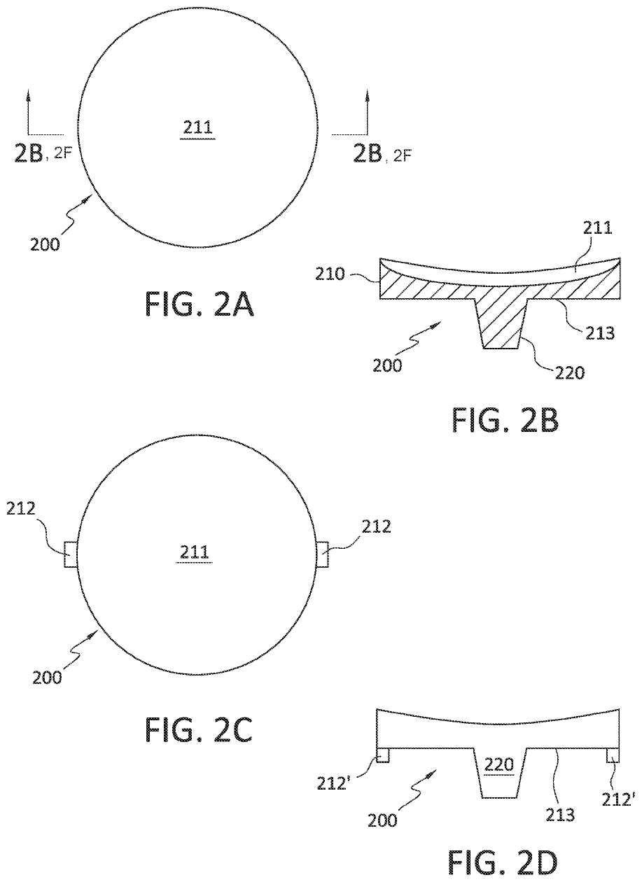

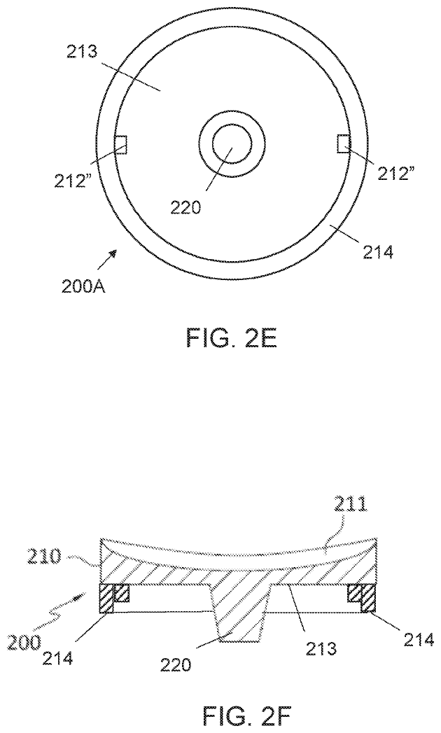

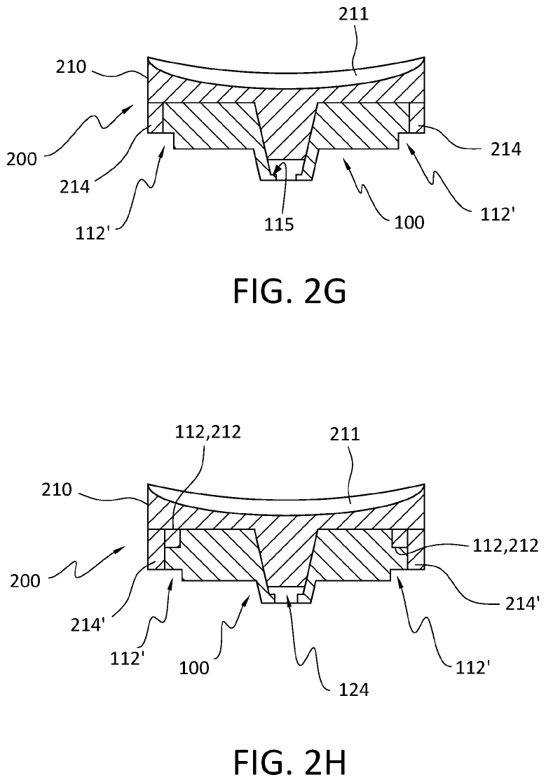

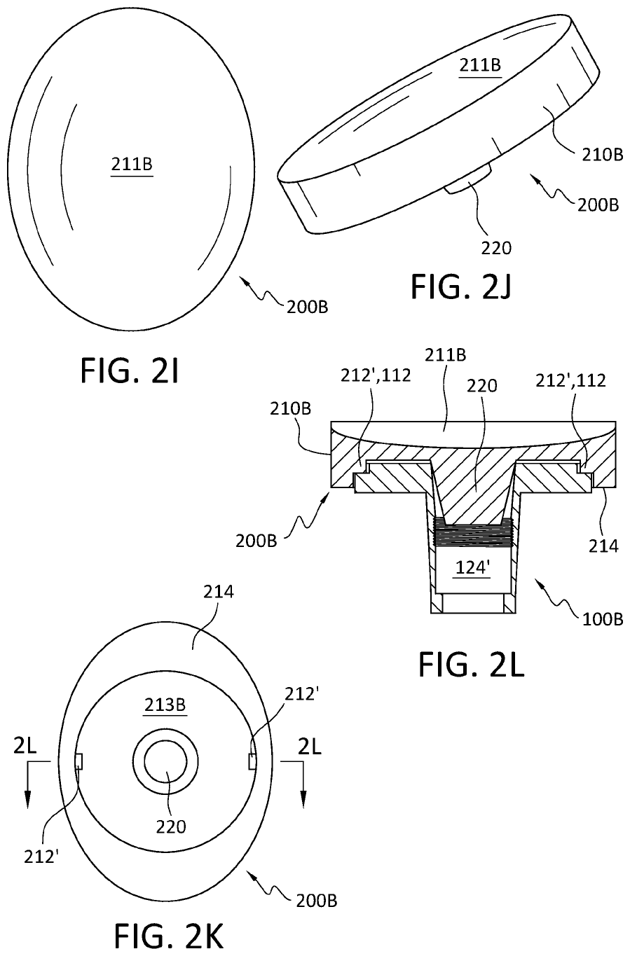

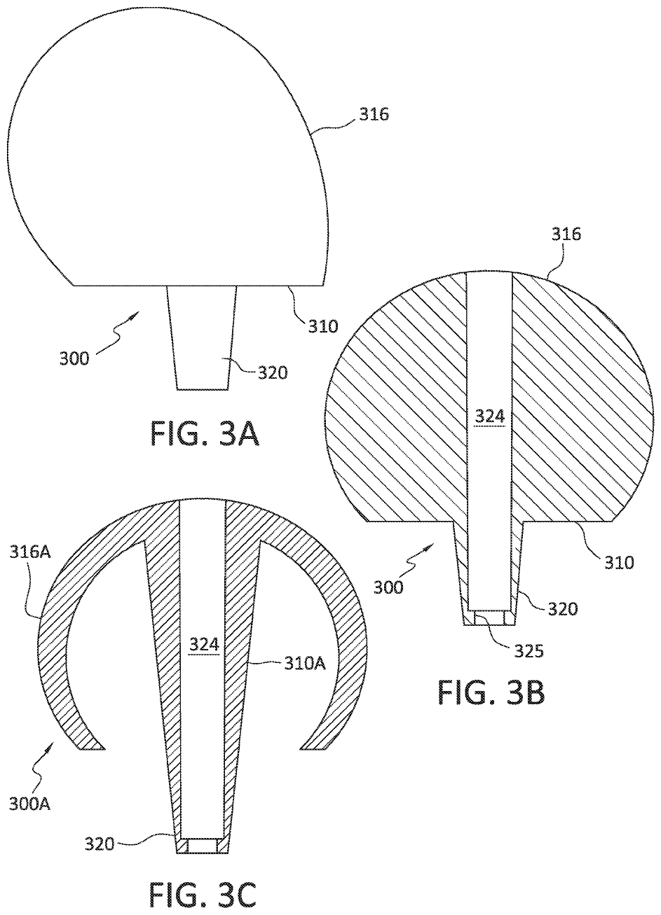

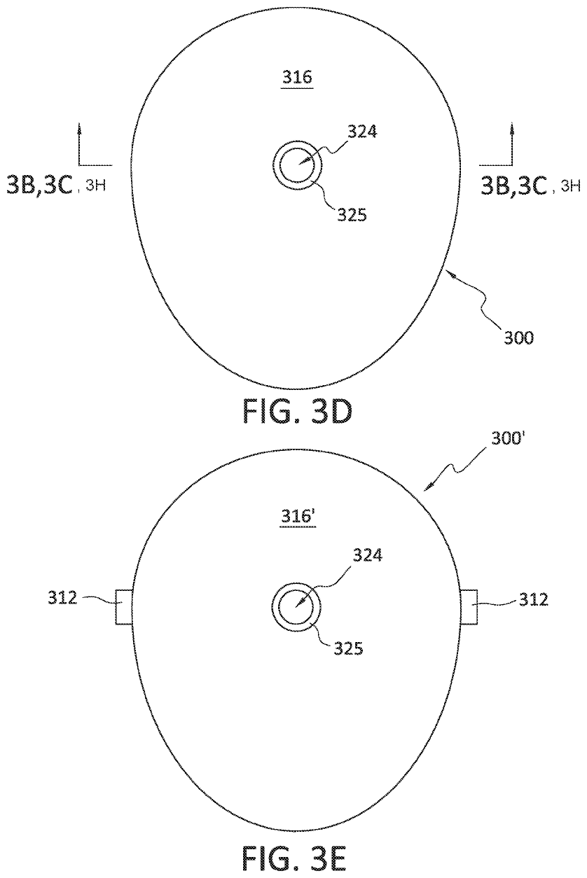

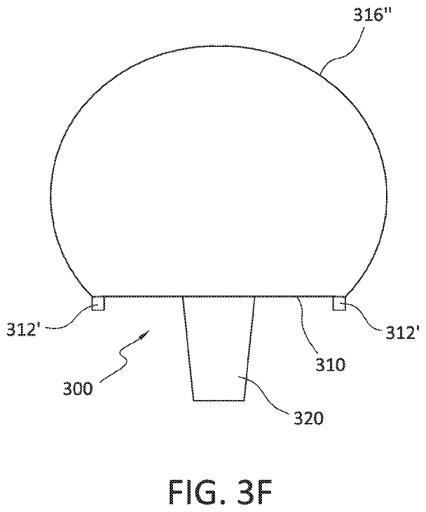

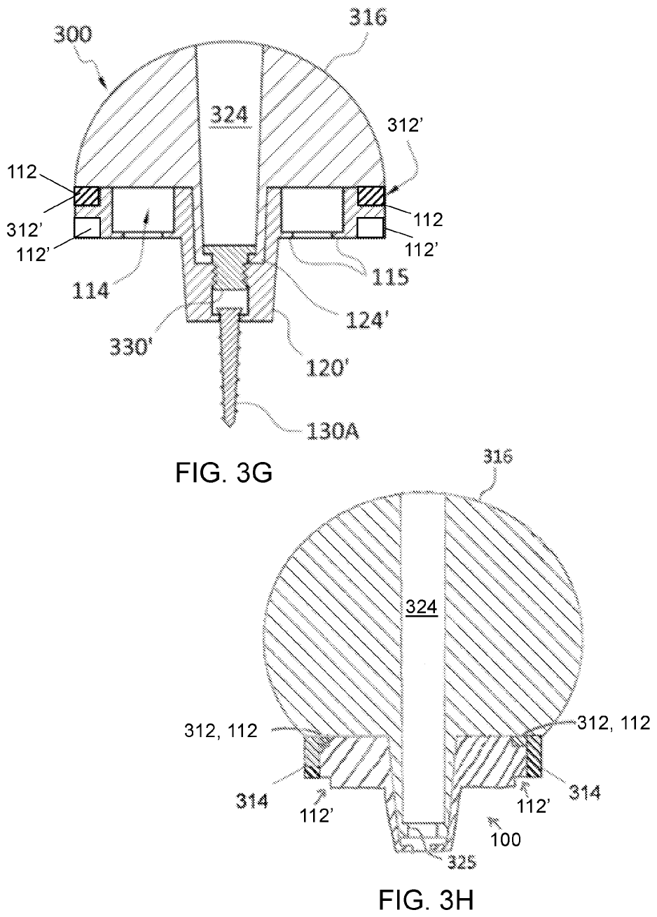

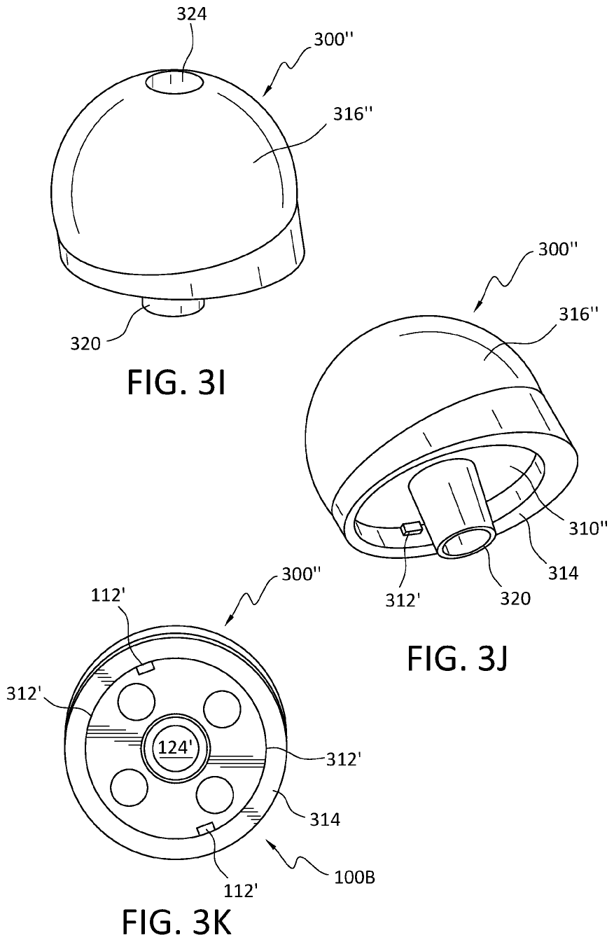

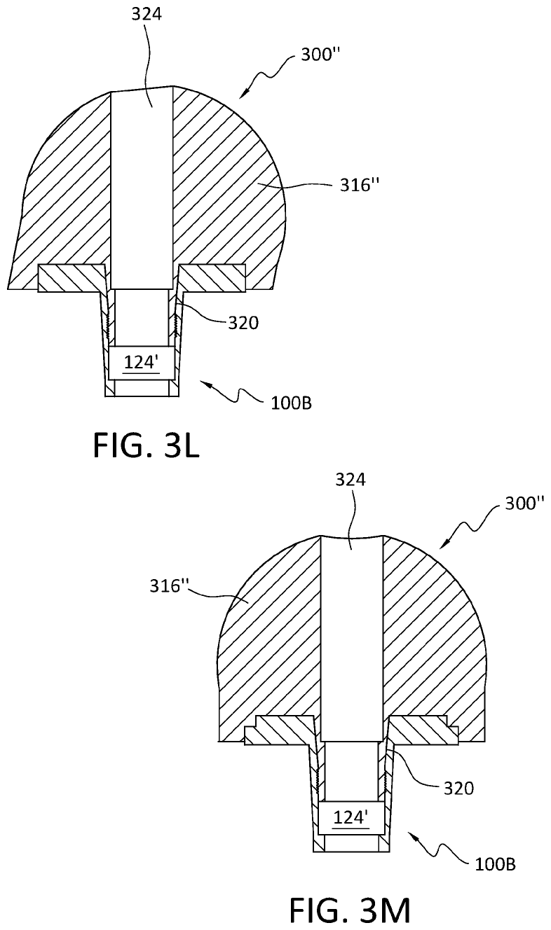

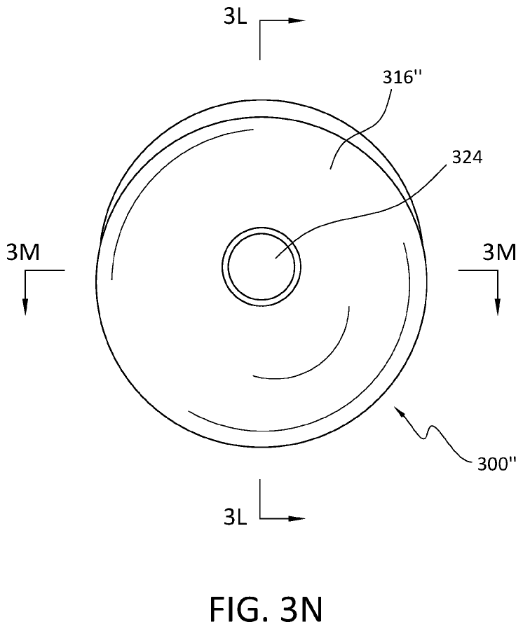

The illustrated base 110 of FIGS. 1A-1C includes a pair of opposed leverage notches 112, 112 extending down from outer circumferential sides of the mounting surface 111, for example on the anterior and posterior central exterior edge (or side) of the base 110, which in at least one embodiment provides better access to the notches for removal of the modular component. The notches 112, 112 are configured to be accessible from the mounting surface 111. In at least one embodiment, the notches 112, 112 provide (or are configured to have) a leverage point to facilitate separation of the mounted modular component from the baseplate 100 when the baseplate 100 is implanted. The notches 112, 112 have sufficient width and depth to receive an instrument in which to pry the mounted modular component from the baseplate 100. Although two notches 112, 112 are illustrated, additional notches could be added to the base 110. In a further embodiment, each of the notches 112, 112 receive a protrusion 212′/312′, 212′/312′ (see, e.g., FIGS. 2D, 2H, 3F, and 3G) depending from a base of the modular component, for example to increase the strength of the connection between the base 110 and the modular component 200, 300. In at least one embodiment when additional notches are present, the modular component may be rotated relative to the base to engage the two notches that provide a preferred orientation for engagement of the modular humeral component by the modular component. In a further embodiment, a space will be defined by the notch 112 and the protrusion 212′/312′ to receive an instrument to pry the mounted modular component 200, 300 from the baseplate 100. In at least one embodiment, the leverage notches 112, 112 are omitted, while in another embodiment the leverage notches are the attachment points for fingers of the instrument.

In at least one embodiment where leverage notches are present, the leverage notches and the attachment points are aligned with each other as illustrated, for example, in FIG. 1F, while in an alternative embodiment the leverage notches and the attachment points are offset from each other as illustrated in FIGS. 1G-1J.

The illustrated base 110 includes five mounting holes 114A-114E with an axially centered hole 114A and four evenly spaced perimeter holes 114B-114E around the mounting surface 111. The holes 114A-114E are illustrated as having a shoulder 115 on which a screwhead, which is an example of an attachment mechanism 130, 130A, will make contact after insertion into the baseplate 100. Although five holes are illustrated, the number of holes could be reduced, including omission of the perimeter holes, or increased. In at least one embodiment, the central hole 114A defines a chamber 124 for receiving a modular component plug. In at least one embodiment, the holes 114B-114E are offset from the notches 112, 112 as illustrated, for example, in FIG. 1A. For example, hole 114B might be at approximately 1 or 2 o'clock while hole 114E might be at approximately 10 or 11 o'clock if the notches 112, 112 are at 3 and 9 o'clock, respectively. FIG. 1G illustrates an alternative embodiment where the openings 114C and 114E are not offset with notches 112, 112, 112′, 112′.

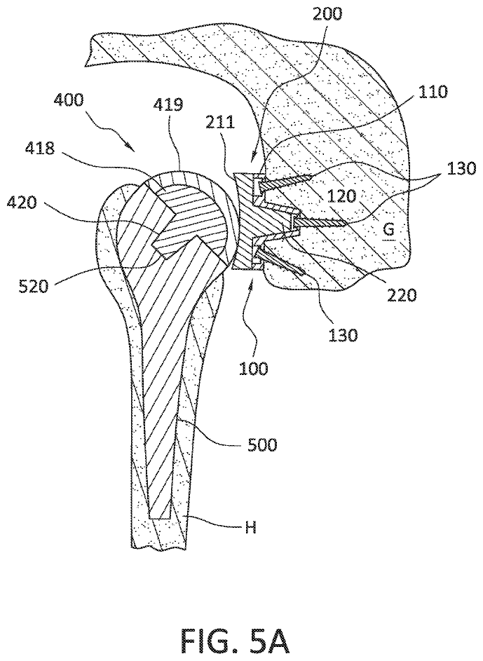

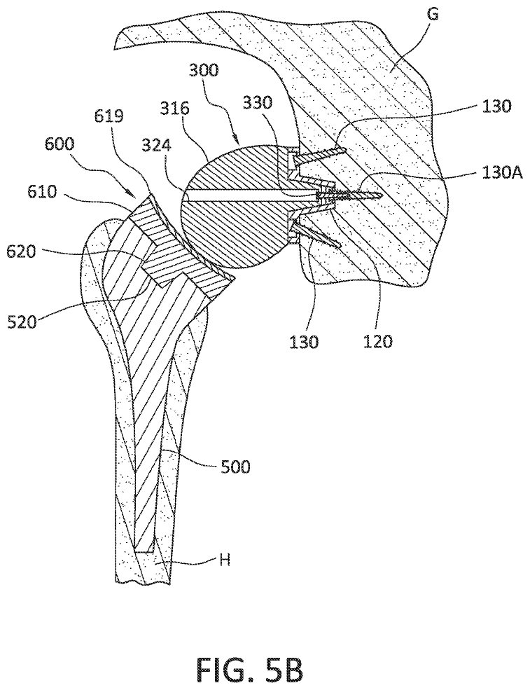

In at least one embodiment, one or more variable angle locking screws 130, 130A are used to attach the baseplate 100 to the patient's glenoid bone G. FIGS. 5A and 5B illustrate the use of screws 130, 130A anchoring the baseplate 100 to the glenoid bone G. Examples of screw diameters include 4.5 mm to 5.0 mm and in a further example including the end points of that range, and more particularly 5.0 mm. Although there are five holes illustrated, during a particular procedure, all five holes may not be utilized. In at least one embodiment, the flexibility in which holes 114A-114E to use and the variable angle locking screws 130, 130A provides flexibility to the orthopedic surgeon in securing the baseplate 100 to the patient's glenoid bone G. Examples of locking screw angles includes between 20 degrees and 30 degrees (with or without the end points) or perpendicular to the base 110. FIGS. 1B and 5B illustrate a screw 130A that includes a receiving cavity 134 for insertion of a torque limiting fastener inserted through the modular component.

In at least one embodiment, the central axial opening 114A passes from the base 110 into and through the stem 120 to allow for the top of the locking screw 130A to be deeper into the baseplate 100 and to provide the chamber 124′ for receiving a plug, e.g., the Morse taper, of the modular component being mounted onto the baseplate 100. FIG. 1C illustrates the chamber 124′ having receiving screw threads for engaging a torque limiting fastener 330′. As referenced above, the torque limiting fastener may engage locking screw 130A instead or in addition to other areas of the chamber 124′. FIG. 1C illustrates the modular component as the glenosphere component 300, 300′ discussed in connection with FIGS. 3A-3F. In a further alternative embodiment, the modular component may include protrusions depending down from the bottom surface configured to have an interference fit with one or more of openings 114B-114E similar to the plug be received in chamber 124/124′.

FIGS. 1D and 1E illustrate a pair of alternative baseplates 100A, 100B that have a partial wedge 116A and a full wedge 116B, respectively. One of ordinary skill in the art should appreciate that the presence of a wedge is potentially advantageous for addressing bone deformities of the glenoid while providing a secure attachment of the baseplate 100 to the patient's glenoid bone G. Alternatively, the baseplate 100 can have a 10 degree or 25 degree back as oppose to the neutral back illustrated in FIG. 1B. In embodiments with a wedge, the attachment points may be a combination of a slot 112″ on one side and a notch 112′ on the opposing side to the wedge may be added to the baseplates 100A, 100B. Also illustrated in FIGS. 1D and 1E are optional notches 112, 112.

FIGS. 1G-1I illustrate an alternative baseplate 100C with notches 112, 122 at approximately 3 and 9 o'clock, respectively, and attachment points 112′, 112′ at approximately 5 and 11 o'clock, respectively. FIG. 1J illustrates the presence of a threaded section 1242′ present in chamber 124′ of the plug 120. Otherwise, FIGS. 1G-1I mirror the previously discussed FIGS. 1A-1C and 1F.

FIGS. 2A (top view) and 2B (cross-section view) illustrate a modular glenoid component 200 having a concave surface 211 on a base 210 for receiving a prosthetic ball, spherical object, or another convex shaped interface and a plug 220 extending from a surface 213 opposed to the concave surface 211 on a base 210. One of ordinary skill in the art should appreciate based on this disclosure, the concave surface can take a variety of shapes without departing from the modularity of the modular glenoid component 200. An example of a different shape is a variating surface with a non-uniform level of curvature over the concave surface. The plug 220 is configured to be inserted and frictionally engage the chamber 124 in the baseplate 100. In at least one embodiment, the plug 220 is a Morse taper central plug.

Although the modular glenoid component 200 is illustrated as being round in FIGS. 2A and 2C, the concave surface 211 may be elliptical, oval or other similar shapes when viewed from the top as illustrated in FIGS. 2I-2L.

In a further embodiment illustrated in FIG. 2C, the modular glenoid component 200 includes a pair of protrusions 212, 212 extending radially out from the anterior and posterior edges to provide additional surface area on which to apply leverage to remove the modular glenoid component 200 from the baseplate 100. In at least one embodiment, the protrusions 212, 212 are aligned with the notches 112, 112 after the modular glenoid component 200 is implanted. In a further alternative embodiment, the protrusions 212′, 212′ depend from the bottom surface 213 as illustrated in FIG. 2D. Protrusions 212′, 212′ are shaped to be inserted into and engage at least a portion of a corresponding notch 112. In at least one further embodiment, the protrusion 212′ and the notch 112 have an interference fit. As discussed above, the notch 112 and the protrusion 212′ may define a space for receiving an instrument, or alternatively the protrusion 212′ fully fills the space of the notch 112. In another alternative embodiment, the protrusions 212, 212′ illustrated in FIGS. 2C and 2D, respectively, are combined together. In at least one embodiment where the protrusions 212, 212′ are present and the leverage notch is also the attachment point, fingers of the instrument will reach below at least one protrusion to engage the modular component. In a further embodiment, the finger will have a step in it such to engage the space between flange 212′ and the notch 112 with a step to then engage the flange 212 to provide additional leverage. In a further alternative embodiment with a flange for the modular component, the protrusion 212 extends radially out from the peripheral side of the base and/or the flange.

FIGS. 2E-2G illustrate an alternative modular glenoid component 200A that includes a flange 214. FIG. 2E illustrates a bottom view of the modular glenoid component 200A with the flange 214 and the plug 220. The flange 214 extends down from bottom surface 213 and the outer circumferential edge of the modular glenoid component 200A. The flange 214 is configured to fit over and down at least a portion of the peripheral external sides of the baseplate 100. In this embodiment, the base 210 has a wider diameter than the base 110 of the baseplate 100. FIGS. 2E and 2F also illustrates an optional pair of protrusions 212″ that are located on the inside surface of the flange 214. These protrusions 212″ are similar to protrusions 212′ discussed above. In at least one embodiment, the flange 214 includes at least two slots extending up from the bottom of the flange 214 toward the base 210, which in at least one embodiment are configured to align with the attachment points of the baseplate.

FIG. 2G illustrates a modular component 200 with a flange 214 attached to a baseplate 100. Although the bottom of the flange 214 is shown as being spaced from the bottom outer edge of the baseplate 100, these surfaces may be flushed with each other.

FIG. 2H illustrates a cross-section with protrusions 212′, 212′ extending down from the base 210 of the modular component 200 and on the inside of the flange 214. FIG. 2H also illustrates the presence of a protrusion 212′ engaging the notch 112.

FIGS. 2I-2L illustrate an alternative modular component 200B with an elliptical shaped concave surface 211B. FIGS. 2K and 2L illustrate the presence of a flange 214 extending down from the bottom surface 213B of the base 210B and a pair of protrusions 212′ extending down from the base 210B and in from the flange 214. In at least one embodiment, the flange 214 is the remaining body of the glenoid component when the central circle equal to the baseplate is cut-away (or otherwise removed) to allow for the glenoid component to interlock and seat onto the baseplate. Although the concave surface is elliptical, the cavity defined by the flange 214 is configured to fit over the base plate 100B as illustrated in FIG. 2L. FIG. 2L also illustrates the engagement of the protrusions 212′ into the notches 112. Although not illustrated, the post 220 could be cylindrical shape to provide additional interference fit with the chamber 124′.

In at least one embodiment, the modular glenoid component 200 is manufactured from Cobalt-Chromium (Co—Cr) to improve the life expectancy for the implant.

FIGS. 3A-3C illustrate a glenosphere component 300, 300A with a glenosphere 316 extending from a base 310 and a plug 320 extending from a baseplate engagement face of the base 310. In at least one embodiment, the plug 320 of the glenosphere component 300, 300A is similar to the plug 220 discussed above in connection with the modular glenoid component 200 including any further or alternative embodiments.

In at least one embodiment, there is a 7-10 degree inferior tilt (see, e.g., FIGS. 3A, 3D, 3E, and 3I) added to the glenosphere that allows for improved glenosphere positioning for particular patient situations. In a further embodiment, the inferior tilt is approximately 10 degrees. “Approximately” is being used here to accommodate manufacturing tolerances of 2 or fewer degrees. The inferior tilt is measured from a reference axis that extends through the base 310 and is perpendicular to the baseplate engagement face of the base, which is the face that abuts against the baseplate. When the glenosphere is implanted onto a baseplate, the reference axis will be substantially parallel to the ground and horizontal using this orientation. In at least one embodiment, the reference axis is centered with the plug 220. There are a major axis and a minor axis that intersect at the reference axis. The minor axis is from the anterior side to the posterior side, while the major axis is perpendicular to the minor axis similar to the definition of a major axis for an ellipse. In at least one embodiment, the optional passageway and plug 220 will be centered as to the minor axis (cross-section plane in FIG. 3N for FIG. 3M) (see, e.g., FIG. 3M), but off-centered along the major axis (cross-section plane in FIG. 3N for FIG. 3L) when viewed from the anterior side or the posterior side (see, e.g., FIGS. 3L and 5B).

In a further embodiment, the inferior mass of the glenosphere protrudes from the base at an angle by approximately 7 to approximately 10 degrees inferiorly from the reference axis. The presence of an inferior tilt in the glenosphere avoids the need to remove the baseplate and to remove additional glenoid bone to provide a suitable angle for engagement of a non-tilted glenosphere with a humeral cup. In at least one embodiment, the majority of the mass of the glenosphere is on the inferior side of the optional passageway 324 thus providing a spherical area aligned with a humeral cup and providing a closer approximation to the natural movement of the humerus relative to the glenoid. When the glenosphere is viewed from the side after implanting like the view depicted in FIG. 5B, the majority of the mass is below the optional passageway 324 (or alternatively a horizontal axis passing through the axial center of the baseplate 100). In a further embodiment, the length of the glenosphere is shorter than that depicted in the figures (i.e., the distance from the engagement face for the baseplate to the opposing surface of the glenosphere (the left side of the glenosphere in FIG. 5B or the top of the glenosphere in FIGS. 3A-3C, 3F-3J, 3L, and 3M)) while maintaining a generally oblong look to it. In a further embodiment, a cross-section of the glenosphere is similar to a stylized single quote.

In at least one embodiment, the center of rotation (COR) for the glenosphere will have a 10 mm lateralization of the COR with a diameter of 32 mm. In a further embodiment, the “minus” sizes in 4 mm increments will allow for a 6 mm lateralization to diminish glenoid bone-prosthesis surface shear stresses. Examples of this concept include glenosphere diameters of 32 mm, 36 mm, and 40 mm with minus options to keep lateralization to 10 mm or 6 mm such that 32 mm neutral and 32 mm “−4”, 36 mm (is already a “−4”) and a 36 mm “−4” (which results in a “−8”), 40 mm (by definition is a “−8”) and a 40 mm “−4” (which results in a “−12”).

The shape of the glenosphere may vary from that illustrated and the shape illustrated in these figures are not intended to limit the exact shape as these are illustrative figures. More particularly, FIG. 3A illustrates a view taken anteriorly (i.e., standing in front of the right shoulder when implanted).

FIGS. 3B-3D illustrate an embodiment that includes a passageway 324 passing through the axial center of the glenosphere 316, 316A and the plug 320. In at least one embodiment, the passageway 324 receives a fastener (not illustrated) to further secure the modular glenoid component 300 to the baseplate 100. In a further embodiment, the passageway includes a shoulder 325 on which a screwhead will make contact after insertion into the passageway 324.

FIGS. 3B and 3C illustrate alternative embodiments for the glenosphere that both illustrate the optional passageway 324. FIG. 3B illustrates a substantially solid glenosphere 316 other than the optional passageway 324. FIG. 3C illustrates a glenosphere 316A that is hollow other than an exterior surface and the base 310A that includes the plug 320 in this embodiment. The illustrated base 310A includes a support column on which the glenosphere 316A is supported. In at least one embodiment, the glenosphere 316A in FIG. 3C extends down a sufficient distance to overlap with the external peripheral sides of the baseplate 100 with the extended length overlapping the baseplate sides and is an example of a flange.

In a further embodiment illustrated in FIG. 3E, the modular glenoid component 300′ includes a pair of protrusions 312, 312 extending radially out from the anterior and posterior edges of the glenosphere 316′ to provide additional surface area on which to apply leverage to remove the modular glenoid component 300′ from the baseplate 100. In a further alternative embodiment, the protrusions 312′, 312′ depend from the glenosphere 316″ when viewed from the top (or bottom) side as illustrated in FIGS. 3F and 3G and similar to protrusions 212′, 212′ discussed above in connection with the modular glenoid component 200 including alternative embodiments, hybrid protrusion embodiments, and engagement between protrusion and notch. FIG. 3G illustrates an example of how the protrusions 312′, 312′ would engage the notches 112, 112, of the baseplate 100. Based on this disclosure, it should be understood that the protrusions 312, 312′ may extend from the glenosphere 316 or 316A. The instrument for implantation and extraction, depending on the embodiment, will have fingers to match protrusions 312 and/or 312′ as described above in connection with protrusions 212, 212′.

FIG. 3H illustrates an alternative modular glenoid component 300B that includes a flange 314 extending from the bottom of the base 310. The flange 314 extends down from bottom surface and the outer circumferential edge of the base 310. The flange 314 is configured to fit over and down at least a portion of the peripheral external sides of the baseplate 100. In this embodiment, the base 310 has a wider diameter than the base 110 of the baseplate 100. Although not illustrated, the protrusions 312′, discussed above, may be present on the inside surface of the flange 314. In at least one embodiment, the flange 314 includes at least two slots extending up from the bottom of the flange 314 toward the base 310, which in at least one embodiment are configured to align with the attachment points of the baseplate.

FIGS. 3I-3M illustrate another alternative glenosphere embodiment with FIGS. 3K-3M illustrating it attached to a baseplate 100B. The line passing through the glenosphere in these figures is not representative of a break between materials or different components. The illustrated modular glenosphere component 300″ includes a glenosphere 316″ having an inferior tilt of approximately 10 degrees from the vertical axis perpendicular to the base 310″. As illustrated in FIGS. 3J and 3K, there are a pair of protrusions 312″ extending down from the base 310″ and in from a flange 314. In at least one embodiment, the posterior and anterior sides of the glenosphere are substantially parallel to the vertical axis over a majority of the respective height of the sides. FIGS. 3I-3L also illustrate how the plug 320 and the passageway 324 are offset eccentrically from the glenosphere 316″. FIGS. 3L and 3M illustrate how in at least one embodiment the top of the opening for passageway 324 is at the proximate top of the glenosphere 316″. The cross-sections in FIGS. 3L and 3M are taking along different diameters passing through the glenosphere 316″ as illustrated in FIG. 3N. FIG. 3N also illustrates a top view of the glenosphere component 300″ illustrating how the passageway 324 is centered relative to the anterior and posterior sides of the glenosphere 316″ and off centered relative to the inferior and its opposite side when viewed from the top.

In a further embodiment, the glenosphere may have a surface area having an arc extending for approximately 180 degrees to approximately 270 degrees. In a further embodiment, the glenosphere is approximately a three-quarters oblong sphere and/or hemi-spherical.

In at least one embodiment, the glenosphere 316 is made from Co—Cr.

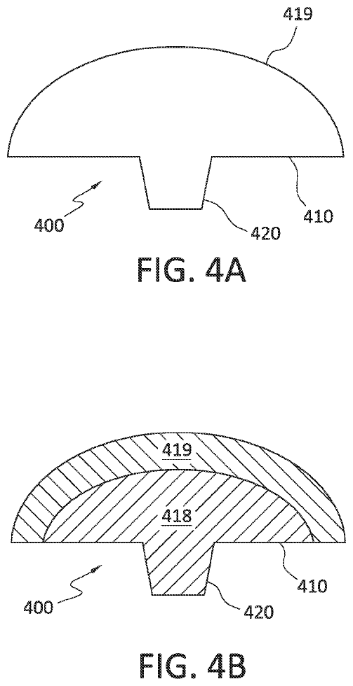

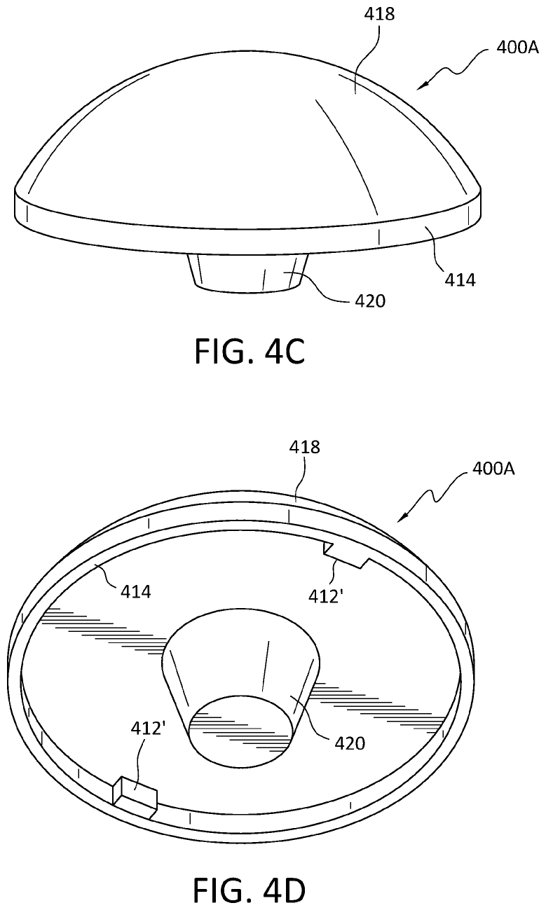

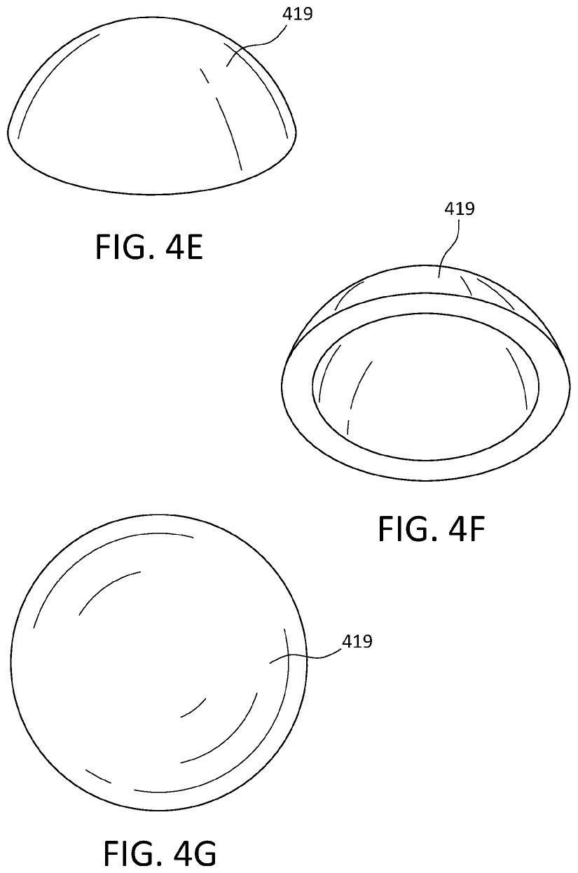

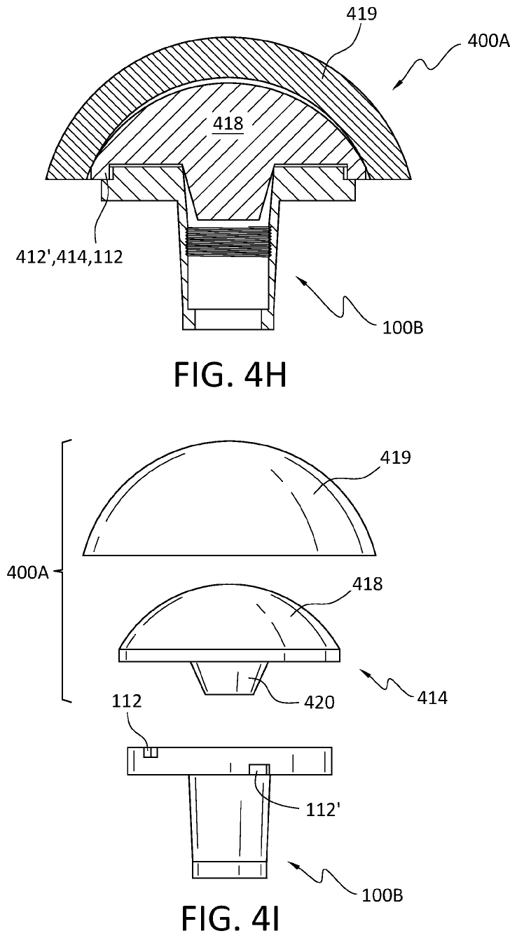

FIGS. 4A-4I illustrate different dual mobility humeral heads having an outer shell (or cap) configured to slide relative about an inner head (or dome). The relative sliding between these two components includes rotational and/or along an arc. In at least one embodiment, the cap and the dome are hemi-spherical.

FIGS. 4A (side view) and 4B (cross-section view) illustrate a dual mobility humeral head 400 for a ta-TSR. The illustrated humeral head 400 includes an outer shell 419 that is pressed over an inner head 418 of a base 410 and a plug 420 for insertion into a receiving cavity 520 of a humeral stem 500 embedded into the patient's humeral bone H as illustrated in FIG. 5A. The outer shell 419 is sized to interact with the concave surface 211 of the modular glenoid component 200 as illustrated in FIG. 5A. In at least one embodiment, the inner head 418 and the outer shell 419 are hemi-spherical domes. In at least one embodiment, the outer shell 419 is configured to slide relative to the inner head 418. In a further embodiment, the outer shell 419 is smaller than an exterior surface of the inner head 418 to provide additional range of dual-mobility.

In at least one embodiment, the inner head 418 will be made from Co—Cr while the outer shell 419 will be made from high-density polyethylene, which will avoid the issue of having metal components rub against other metal components, which could lead to a faster wear on the components and potentially create loose metal shavings within the shoulder socket. In an alternative embodiment, the outer shell 419 is removable from the inner head 418 to facilitate replacement, for example when the outer shell 419 is worn down from biomechanical movement about the shoulder joint.

In at least one embodiment, the humeral head 400 will have a centrally located plug 420 that is capable of being manually offset or is physically offset from the axial center to allow best coverage of the proximal humeral anatomic neck and metaphysis. The physical offset includes an eccentrically located plug 420, which when the modular humeral component is rotated provides different amounts of coverage to the humeral side.

FIGS. 4C-4H illustrate an alternative embodiment for the humeral head 400A with FIGS. 4H and 4I illustrating the humeral head 400A having an inner head 418 and a dual mobility outer shell (or head or cap) 419 attached to the baseplate 100B. FIGS. 4C and 4D illustrate inner head 418 while FIGS. 4E and 4F illustrate the outer shell 419 with FIG. 4G illustrating a top view of the outer shell 419 of the humeral head 400A. As illustrated in FIG. 4D, the humeral head 400A includes a flange 414 and an optional protrusion 412′, which is configured to engage notch 112 of the baseplate 100. Although FIG. 4H illustrates the flange 414 not extending down the sides of the baseplate 100B, in at least one embodiment the flange 414 will extend further down the peripheral side of the baseplate 100.

In at least one embodiment, the outer shell 419 and/or the inner head 418 will include protrusions similar to the protrusions 212, 212′, 312, 312′ discussed above when the humeral base includes leverage notches to allow for an instrument to be used to pry the outer shell 419 and/or the inner head 418 from the humeral base.

FIG. 5A illustrates an example of how the modular humeral component, which is illustrated as the dual-mobility humeral head 400, cooperates with the modular component, which is illustrated as the modular glenoid component 200. The humeral head 400 is designed to fit into the concave surface 211 of the modular glenoid component 200, which for example is due to similar radiuses of curvature of both sides—the humeral head and the glenoid in a traditional TSR.

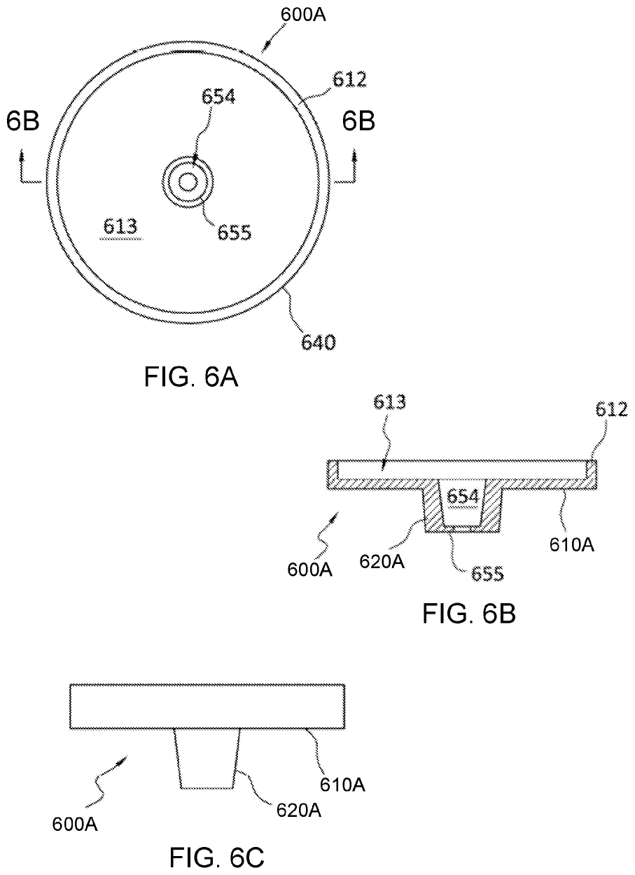

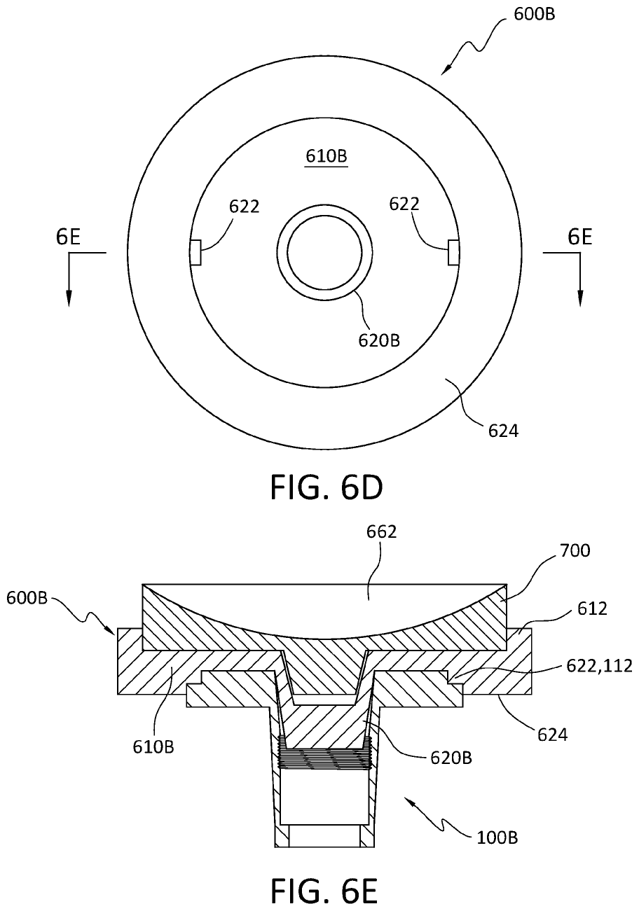

FIG. 5B illustrates an example of attachment of a humeral cup 600 as the modular humeral component to the humeral stem 500 while engaging with a glenosphere component 300 as the modular glenoid component to the baseplate 100. The humeral cup 600 is illustrated as having a shell 619 over a concave surface 611 (see, e.g., FIG. 6B) of a floor 610 from which a plug 620 extends for engagement with the humeral stem 500. In an alternative embodiment, the glenosphere is approximately three-quarters spherical.

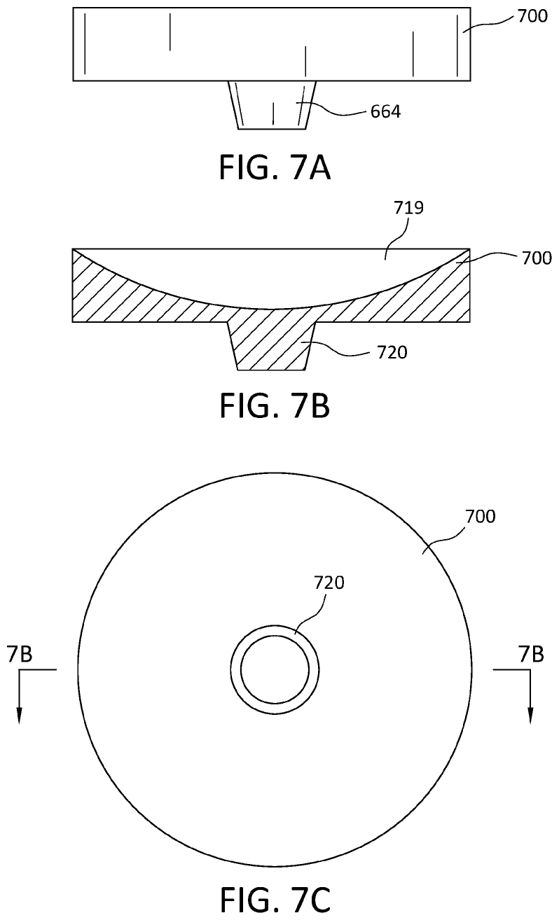

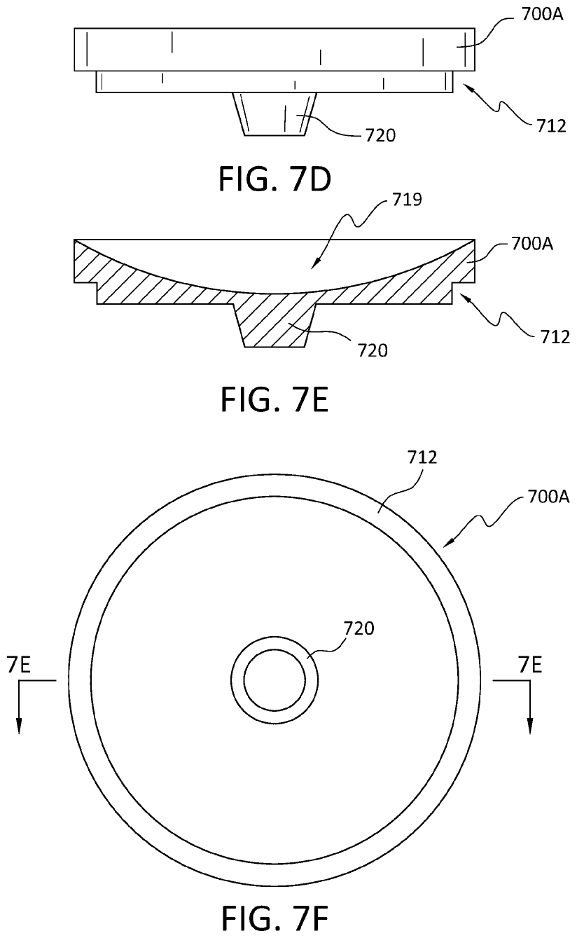

FIGS. 6A-7F illustrate different example components that may be used as part of a humeral cup for a r-TSR, which in at least one embodiment could be used on the glenoid side in a TSR. FIGS. 6A-6E illustrate a pair of example humeral cup bases 600A and 600B, while FIGS. 6E-7F illustrate a pair of example humeral cup shells 700 and 700A. In at least one embodiment, the humeral cup shell may be replaced by a coating over a concave surface on the humeral cup base as illustrated in FIG. 5B. The concave surface is adapted to interact with a glenosphere component 300. In an alternative embodiment, the concave surface includes a variating surface with a non-uniform level of curvature over the concave surface. In at least one embodiment the humeral cup will be made from Co—Cr, for example when the glenosphere component 300 has a high-density polyethylene cover, layer, and/or coating on it.

In at least one embodiment, the humeral cup base 600A, 600B is made of Co—Cr or a similar metal to the humeral stem 500 (including a compatible metal) while the humeral cup shell (or shell) 700, 700A is made from high-density polyethylene or related articulating material. FIGS. 6A and 6B illustrate the humeral cup 600A with a shallow cylinder 612, for example with a height of 4 mm to 8 mm, a diameter of 30 mm, 35 mm or 40 mm to hold the various diameter humeral cup shells 700, 700A that are inserted into a receiving cavity 613 defined by walls 612 that extend up from the floor 610A.

In at least one embodiment illustrated in FIGS. 6B and 6C, the floor 610A includes a centrally located plug 620A. In at least one embodiment, the plug 620 has a Morse taper to engage the humeral stem 500. In at least one further embodiment, the plug 620A is capable of being manually offset or is physically offset from the axial center to allow best coverage of the proximal humeral anatomic neck and metaphysis. The physical offset includes an eccentrically located plug 620, which when the modular humeral component is rotated provides different amounts of coverage to the humeral side.

In at least one embodiment, the humeral cup base 600A includes a passageway 654 passing from the bottom of the shell receiving cavity (or chamber) 613 through the plug 620A, which may receive a stem of the inserted shell 700, 700A such as those illustrated in FIGS. 7A-7F. In at least one embodiment, the receiving cavity 613 is short with the shell fitting into the receiving cavity 613, and in a further embodiment such as illustrated in FIG. 6E, the shell extends beyond the top of the receiving cavity 613 away from the mounting floor 610A. The passageway 654 may include a shoulder 655 around an opening to facilitate the use of an attachment mechanism as illustrated in FIGS. 6A and 6B, which opening may be omitted as illustrated in FIG. 5B.

In at least one embodiment, the shell is press-fit into the receiving cavity 613. In at least one embodiment, the shell 700, 700A includes a curvature to it that has a uniform thickness, but in other embodiments the central portion is thicker than the edges. In a further embodiment, when the shell has been worn down, then it is removed and replaced. One approach for removing the shell is prying the shell 700, 700A from the receiving cavity 613; another approach for removing the shell is to freeze it with liquid Nitrogen to shrink it before it pops out from the receiving cavity 613.

In at least one embodiment in FIGS. 6D and 6E, the humeral cup base 600B will include protrusions 622. 622 similar to the protrusions 212, 212′, 312, 312′ discussed above when the humeral base includes leverage notches to allow for an instrument to be used to pry the base 600A, 600B from the humeral base. In at least one embodiment as illustrated in FIGS. 6D and 6E, the base 600B may include a flange 624. The flange 624 is designed like the other flanges discussed in this disclosure to extend down and over at least a portion of the outer peripheral wall of the baseplate 100B (or humeral stem) as illustrated in FIG. 6E. FIGS. 6D and 6E also illustrate the optional protrusions 622 extending down from the undersurface of the floor and in from the flange 624.

FIGS. 7A-7C illustrate a humeral cup shell (or shell) 700 that may be inserted into the receiving cavity of the humeral cup base 600A or 600b. As illustrated in FIG. 6E, the humeral cup shell 700 is designed to fit within the cavity of the humeral cup 600A and 600B. The humeral cup shell 700 includes a concave surface 719 for receiving the glenosphere after implantation. The humeral cup shell 700 is illustrated as including an optional plug 720 for insertion into the receiving cavity 654 of the humeral cup 600A or 600B, which in at least one embodiment provides an interference fit.

FIGS. 7D-7F illustrate a humeral cup shell (or shell) 700A that may be inserted into the receiving cavity of the humeral cup 600A or 600B. The humeral cup shell 700A is designed to largely fit within the cavity of the humeral cup 600A, 600B. The illustrated humeral cup shell 700A includes a stepped outer bottom edge (or lip) 712 configured to receive the peripheral outer wall 612 of the humeral cup 600A, 600B. The humeral cup shell 700A includes a concave surface 719 for receiving the glenosphere after implantation. The humeral cup shell 700A is illustrated as including an optional plug 720 for insertion into the receiving cavity 654 of the humeral cup 600A, 600B, which in at least one embodiment provides an interference fit.

In at least one embodiment, the humeral cup shell 700, 700A is made of or includes a coating with polyethylene or a related articulating material.

In at least one embodiment, the stem of either humeral module is impacted onto the humeral stem such as a metaphyseal stem, short stem, or long stem through a variety of fixation techniques including, for example, a reverse Morse taper stem for the cavity 520 accepting the plug 420, 620 of the humeral head/cup 400, 600. Humeral stems known in the art with an adaptation for receiving the plug 420, 620 of the humeral head/cup 400/600 may be used. FIGS. 5A and 5B illustrate an example of a humeral stem 500 anchored in a humeral bone H.

In an alternative embodiment to the above-described modular components, modular glenoid components and modular humeral components (that collectively are examples of modular components) with a central passageway, the central passageway is omitted.

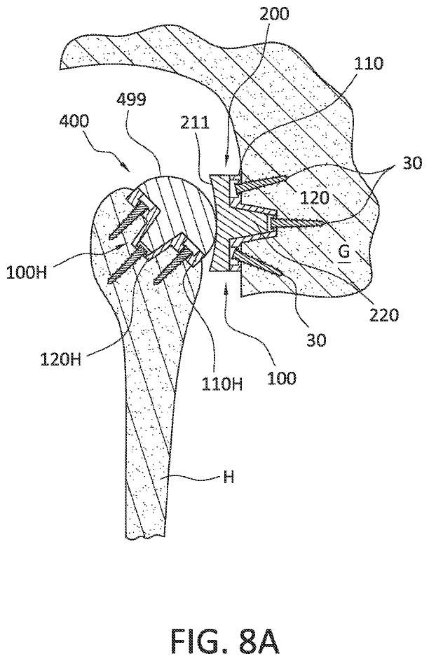

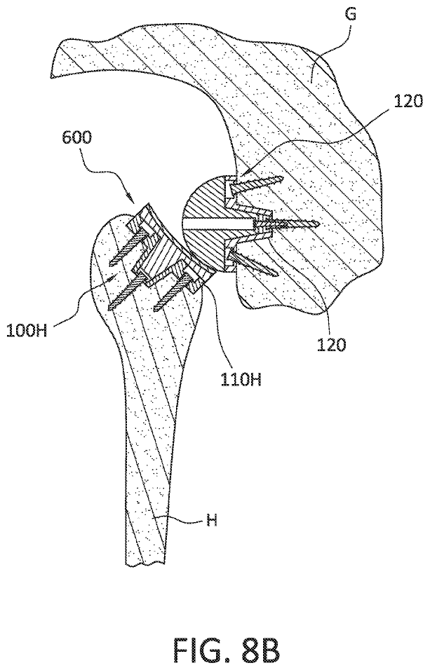

FIGS. 8A and 8B illustrate a configuration where the humeral stem 500 is replaced by a baseplate 100H, which in the illustrated embodiment is a second baseplate, being implanted on the humerus H. The baseplate 100H includes similar structures to the baseplate 100 (including the various above-discussed variants) including a base 110H with a mounting surface and notches and a stem 120H with a receiving cavity. In at least one embodiment, the humeral implanted baseplate 100H is identical to the glenoid implanted baseplate 100. In at least one further embodiment, this configuration allows for interchangeability of modular components between the two baseplates 100, 100H. In another embodiment, the humeral implanted baseplate 100H has a larger diameter for the base 110H and/or the stem 120H, a higher overall height of the base 110H and the stem 120H, a greater thickness of the base 110H, and/or a higher height of the stem 120H than the glenoid implanted baseplate 100. In at least one further embodiment, the modular components to be implanted in the humeral baseplate 100H would have corresponding adjustments. FIG. 8A illustrates an alternative humeral head 400 with an exterior surface 499 that is a one-piece construction for the humeral head 400 without any outer shell.

In at least one embodiment, the humeral stem and the humeral baseplate are examples of the humeral base.

In a further embodiment to any of the embodiments discussed above having protrusions that have an interference fit with notches, the humeral base and/or the baseplate may omit a receiving cavity and instead rely on the interference fit between the protrusions and the notches to secure the attached modular component when the system is implanted in a patient. Examples of the modular component include, for example, those discussed in this disclosure, including a dual mobility humeral component, a humeral cup, a glenoid articular surface, and a glenosphere articular surface.

In an alternative embodiment for the modular components, the modular components include an outer circumferential flange 214, 314, as discussed above, that extends down from the base 210 or the glenosphere 316. In at least one embodiment, the flange is present on the humeral modular component. The base 210 and the glenosphere 316 in this embodiment would have a wider diameter than the baseplate 100 to facilitate the modular component fitting over the baseplate 100. The depth of the flange may be in the range of 1 mm to 10 mm, in the range of 6 mm to 9 mm, or approximately 7.5 mm or 75% of the height of the base. In at least one embodiment, the ranges include the end points. The flange provides a partial to complete overlap along the peripheral wall of the baseplate 100, which in at least one embodiment provides additional engagement between the installed component and the baseplate 100. In at least one embodiment, the leverage notch 112 would have a depth greater than the modular component flange depth to provide a leverage point for removal of the modular component. In a further embodiment, the flange will include two or more slots opening towards the bottom of the flange on which an instrument may engage the modular component for removal from the baseplate. In at least one embodiment, the leverage notches, if present, would have sufficient depth to provide an insertion gap between the top of the flange slot and the bottom of baseplate notch. In a further embodiment, the baseplate notch is wider than the flange slot (or vice versa) to facilitate better alignment of the notch and the slot to each other. In embodiments using a humeral stem, the modular components may have a similar overlap with the humeral stem as with the baseplate. As discussed above, the protrusion may be located on opposed interior sides of the flange for engagement with the leverage notches.

The remaining figures discussed in this disclosure relate to tools according to different embodiments for use with shoulder prosthesis systems including those described above in connection with FIGS. 1A-8B.

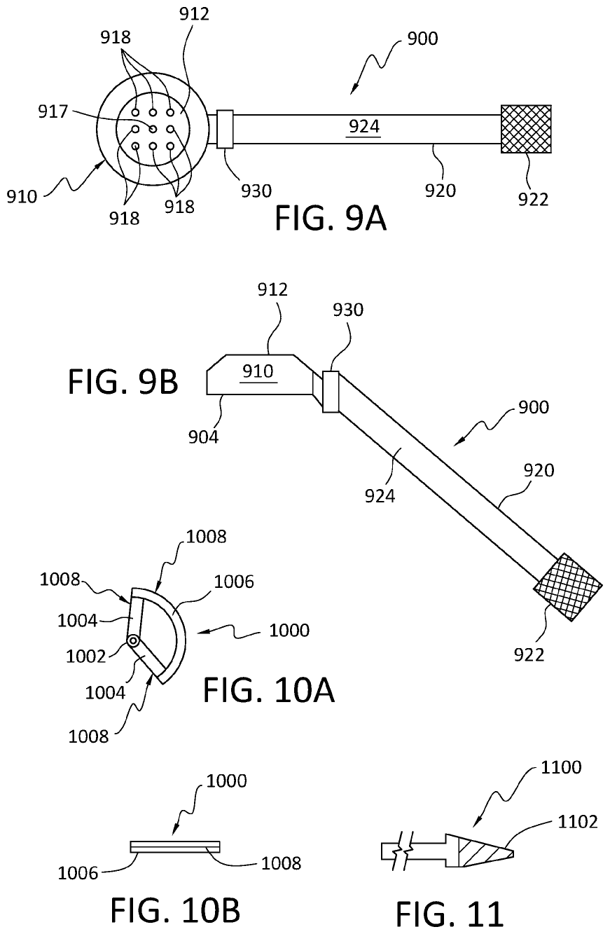

FIGS. 9A and 9B illustrate a drill guide 900 for use in establishing the center hole for the glenoid baseplate. In a further embodiment, the drill guide 900 may be used to establish the location of the mounting hole for the humeral base by flipping (or otherwise positioning) the tool to use a flat surface 914 for placement against the humerus. The illustrated drill guide 900 includes a guide 910 attached to a handle 920 through a locking mechanism 930. The drill guide 900 is used once the glenoid surface has been reshaped or otherwise prepared for the center hole to be drilled.

The illustrated guide 910 includes a fulcrum shaped interface 912 to be placed against the glenoid although this interface surface could instead be flat. The illustrated guide 910 includes a nine-hole array arranged in a 3×3 configuration. In at least one embodiment, the eight offset holes 916 and the center hole 917 are spaced apart by approximately 2 mm gaps measured in the vertical and horizontal distances although other distances may be selected. In at least one embodiment, the guide has a diameter of approximately 15 mm although other diameters may be selected including, for example, those in the range of 14 mm to 20 mm or approximately 19 mm. In a further embodiment, the offset holes 916 form a circle pattern around the center hole 917 instead of the illustrated square pattern. The holes 916, 917 are openings for drill passageways that pass through the thickness of the guide 910.

The locking mechanism 930 sets the angle between the handle 920 and the guide 910. This allows for the guide 910 to rest flush with the glenoid (or the humerus). The handle 920 includes a gnarled knob 922 on the shaft 924 for adjusting the angle to increase the degree to which the guide 910 is flushed against the glenoid (or the humerus). A variety of gnarled patterns may be used on the knob 922 to provide a surface on to which the surgeon can grab. In an alternative embodiment, the handle has a different structure and/or material that still allows the surgeon to manipulate the handle as needed for or during a surgical procedure. The handle 920 also allows the surgeon or another individual to hold the drill guide 900 while a drill bit of approximately 2.5 mm is inserted through the desired hole to establish the center hole location. After the center hole is established, the drill bit may be disconnected from the drill and the guide 910 is slid over the drill bit for removal of the drill guide 900 leaving the drill bit as a guide for other instruments. Although it is possible to remove the drill bit depending on the preference of the surgeon.

The next discussed instrument is a reamer 1000 like that illustrated in FIGS. 10A and 10B. The reamer 1000 includes a center mounting hole 1002 that fits around the anchored drill bit and a pair of radial arms 1004 extending from the center to a cutting face 1006. The cutting face 1006 is approximately a 120-degree arc from the arms forming approximately a 120-degree angle to each other where “approximately” takes into account manufacturing tolerances. In a further embodiment, the arc is in a range of 115 degrees to 125 degrees. The smaller arc is believed to provide for easier insertion and use in the human anatomy confines of the shoulder area than currently used reamers known to the inventor. The radial arms 1004 and the cutting face 1006 include a cutting surface 1008 to define a pocket into which the baseplate 100 (or humeral base) is inserted. The reamer 1000 is engaged by a drill mechanism either electrical or manually powered that allows for the guide drill bit to remain largely in place. After the bone surface has been reamed, the reamer 1000 is removed.

It is possible that the stem drill bit 1100 could be used before the reamer 1000 or afterwards. FIG. 11 illustrates a stem drill bit 1100 for use in expanding the hole into which the baseplate stem 120 will be inserted. Although the stem drill bit 1100 is illustrated as being conical 1102, it may alternatively be cylindrical to match a cylindrical stem. Like with the reamer 1000, the stem drill bit 1100 may also be inserted over the guide drill bit. In at least one embodiment, the stem drill bit 1100 has a length in the range of 6 mm to 7 mm and/or has a diameter that is approximately in the range of approximately 0.5 mm to 1 mm wider and longer than the baseplate stem. In at least one embodiment, the end points of at least one of these ranges are included. In at least one embodiment, the stem drill bit 1100 is either electrically powered or manually powered. After the stem hole is drilled, the stem drill bit 1100 and, if present, the guide drill bit are removed if the reamer 1000 has been used.

FIGS. 12A and 12B illustrate a baseplate inserter 1200, which in at least one embodiment may be used as a baseplate extractor, if needed. The illustrated baseplate inserter 1200 includes a knob handle 1210 engaging a gear mechanism 1220 that engages a pair of arms 1230 extending perpendicularly, radially away from the gear 1220 and the knob handle 1210.

The knob handle 1210 includes a gnarled knob 1212, although the knob handle 1210 may not be gnarled and/or made of different material, with a shaft 1214 that passes through the gear mechanism 1220 and the arms 1230 to a shank 1216 configured to engage the center hole of the baseplate 100. In at least one embodiment, the shaft 1212 includes a threaded surface that engages the gear mechanism 1220 such that as the handle 1210 is lowered into the baseplate 100, the arms 1230 move radially inward to engage the baseplate 100. An example of how the gears may work is a pair of teethed wheels are rotated by the shaft 1214 to then move the arms 1230 in a lateral direction perpendicular to the shaft 1214. In at least one embodiment, the arms 1230 are supported by a guide mechanism, which is part of the gear mechanism in at least one embodiment and is configured to assist with the lateral movement of the arms 1230. The bottom of the shaft 1214 includes the shank 1216, which although illustrated as tapered or having a frustum having complementary beveled sides to match the interior of the chamber 124 of the baseplate 100. Alternatively, the shank 1216 may take a different shape that is able to engage the chamber 124 of the baseplate 100 including a two-stage fulcrum to match the chamber 124′ of FIG. 1C or a cylindrical.

The arms 1230 include a horizontal member 1232, a vertical member 1234 extending down from the horizontal member 1232 in a direction substantially parallel to the shaft 1214, and a cantilever finger 1236 extending from the bottom of the vertical member 1234 radially inward such that the opposed fingers 1236 are aligned with each other for engagement with the attachment points 112, 112′, or 112″ of the baseplate 100. The length of the horizontal and vertical arms 1232, 1234 is based on the design of the baseplate 100 being implanted. In an alternative embodiment, the fingers include a flat gripping surface that with the shank 1216 clamp the baseplate 100 instead of engaging attachment points 112′ or 112″ on the baseplate 100 similar in concept to the way a vise grip (or locking) pliers work.

Together the shank 1216 and the fingers 1236 provide a three-point fixation between the inserter 1200 and the baseplate 100 and provides a fixed alignment between the inserter 1200 and the baseplate 100. Once the baseplate 100 is aligned with the drilled stem hole, the inserter 1200 is configured to be struck with a mallet on the top of the knob 1212 to drive the baseplate 100 into the glenoid (or humerus).

FIGS. 13A and 13B illustrate a baseplate extractor 1300 that is similar to the baseplate inserter 1200 except the ends 1338 of the fingers 1236′ have a cutting edge (or surface) to cut between the baseplate 100 and the bone/tissue interface. The illustrated handle 1210′ illustrates an alternative shaft 1214′ and shank 1216′ from that shown in connection with the inserter 1200. In at least one embodiment, the fingers 1236′ and the vertical arm members 1234′, which are illustrated as having tapered sides along the length of the member, form an angle greater than 90 degrees. The extractor 1300 in use will be rotated around the baseplate 100 to cut the bony ingrowth from beneath and/or sides of the baseplate 100. After the bony ingrowth is cut, the extractor 1300 (or alternatively any of the above-described inserters) is attached to the baseplate 100 and a slap-hammer attachment (or instrument) is used to strike the extractor 1300 to dislodge the baseplate 100 from the bone. In an embodiment where the modular component fits over the sides of the baseplate, the extractor 1300 may be used cut the bony ingrowth from beneath and/or sides of the modular component.

A similar instrument to the above baseplate inserter 1200 and baseplate extractor 1300 may be used for implanting and/or extracting the modular components. In a further embodiment, the shank is omitted and the vertical arms have sufficient length to fit over the modular component interface that engages the counterpart modular component.

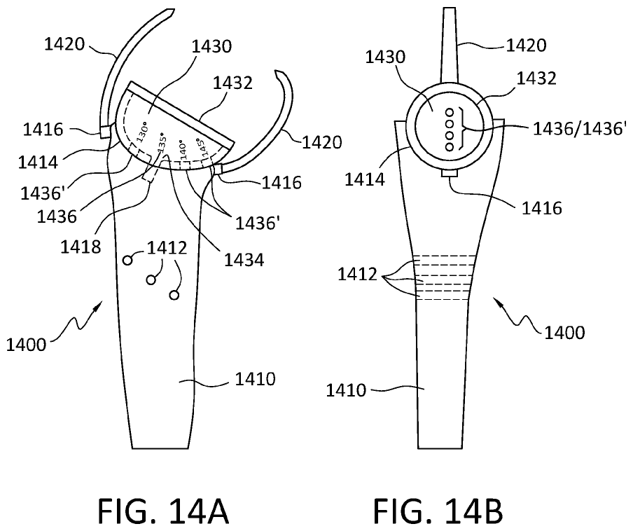

FIGS. 14A and 14B illustrate a humeral neck cutting guide 1400 that provides a flat surface 1432 against which a saw may cut through the humeral neck to provide a surface through which the humeral stem is inserted or on which the humeral baseplate is mounted. The illustrated humeral neck cutting guide 1400 includes a handle 1410 having three optional holes 1412 for placement of alignment rods to determine the degree of retroversion (e.g., 40°, 30°, and 20°) and a receiving cavity 1414, arm slots 1416 on the peripheral exterior edge of the receiving cavity 1414, retractor arms 1420, and a guide 1430 that fits into the receiving cavity 1414.

The guide 1430 allows for the angle of the cut to be set based on the relationship between the guide 1430 and the receiving cavity 1414 with illustrated possible cut angles being 130°, 135°, 140°, and 145° although other angles could be provided for by the guide 1430. In at least one embodiment, the relationship is set by a set fastener 1434 such as a screw or bolt that secures the guide 1430 to the receiving cavity 1414 at the desired angle. In at least one embodiment, the set fastener 1434 passes through a center hole 1436 in the guide 1430 and enters a corresponding hole 1418 in the receiving cavity 1414 for the desired angle. In other embodiments, the set fastener 1434 passes through the desired opening 1436′ for the angle and enters a center hole 1418 in the receiving cavity for the desired angle. In further embodiments to either of the previous two embodiments, the guide may have a series of protrusions for engagement of a plurality of recessions in the receiving cavity.

In at least one embodiment, the retractor arms 1420 are present on the articulate surface and the proximal humeral bone sides of the receiving cavity 1414. The retractor arms 1420 are aligned to provide protection for the rotator cuff, tendons, and neurovascular structures around the humerus by moving these structures away from the bone providing further access for cutting the humerus.

Although particular materials have been identified for particular components and structural elements, one of ordinary skill in the art will appreciate that other materials may be substituted without departing from the scope of the invention. In at least one embodiment, modules attached to the humeral side and the glenoid side will not both be the same material at the point of interaction.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

As used above “substantially,” “generally,” “approximately,” and other words of degree are relative modifiers intended to indicate permissible variation from the characteristic so modified particularly when relating to manufacturing and production tolerances. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

Although one level of multiple dependencies is present in the claims attached to this disclosure, it should be understood that none conflicting claim recitation of dependent claims may be combined, for example, in the manner of the priority applications.

1-61. (canceled)

62. A modular component for use in a shoulder prosthesis system having a baseplate, said modular component comprising:
a base having an upper surface and a lower surface;
a flange extending down from said lower surface along an outer circumferential edge of said lower surface, said flange configured to fit around the baseplate; and
a plug extending down from an axial center of said lower surface, said plug configured to engage the baseplate after implantation in a patient.

63. The modular component according to claim 62, further comprising a pair of protrusions extending down from said lower surface along an interior side of said flange.

64. The modular component according to claim 62, wherein said protrusions are evenly spaced around the interior side of said flange, and/or
said protrusions are configured to engage slots in the baseplate after implantation in a patient.

65. The modular component according to claim 62, wherein said flange includes at least two slots opening downward and extending up from a bottom of said flange towards said base.

66. A modular shoulder prosthesis baseplate comprising:
a base with a plurality of attachment holes passing therethrough from a top surface to a bottom surface and a pair of attachment points on opposed outer circumferential sides of said base, said attachment points extend at least in from said outer circumferential sides of said base, said attachment points are spaced from said top surface; and
a central stem extending from said bottom surface of said base and axially centered with one of said plurality of attachment holes, and
wherein said baseplate is capable of attachment to different modular components to facilitate both traditional anatomic total shoulder replacement and reverse total shoulder replacement with a change in the modular component; and
said attachment points are configured to provide leverage points to facilitate extraction of said baseplate from a patient.

67. The modular shoulder prosthesis baseplate according to claim 66, wherein said attachment points include
at least one notch extending up from said bottom surface of said base, and/or
at least one slot spaced from said bottom surface and said top surface of said base.

68. The system according to claim 66, wherein said baseplate includes a pair of leverage notches on opposed outer circumferential sides of said base, said leverage notches extend down from the top surface of said base and in from said outer circumferential sides of said base.

69. The system according to claim 68, wherein said leverage notches are on the anterior and posterior sides of said base and said attachment points are laterally offset from said leverage notches.

70. A modular shoulder prosthesis system comprising:
a baseplate according to claim 66; and
a modular component configured to be removably attached to said baseplate, said modular component having a plug for insertion into at least one attachment hole of said base.

71. The system according to claim 70, wherein said baseplate includes a pair of leverage notches on opposed outer circumferential sides of said base, said leverage notches extend down from a mounting surface of said baseplate and in from said outer circumferential sides of said base,
said modular component includes a pair of protrusions extending, extending radially, and/or extending down from opposing sides of said modular component, said protrusions configured to engage with an interference fit said leverage notches when said modular component is attached to said base, and a length of said protrusion is less than or equal to a height of said leverage notch, and
said protrusions configured to provide additional surface area on which to apply leverage for removal of said modular component after installation onto said baseplate.

72. The system according to claim 70, wherein said modular component further includes a base and a flange extending down from a bottom of said base, said flange configured to extend down the peripheral sides of said baseplate when said modular component is attached to said baseplate.

73. The system according to claim 72, wherein said flange includes a pair of opposed slots extending up from a bottom of said flange, said slots configured to align with said attachment points of said baseplate when said modular component is attached to said base and to provide a leverage point on which to apply leverage for removal of said modular component after installation onto said baseplate.

74. The system according to claim 70, wherein said modular component includes
a base or cup having a concave surface and a peripheral flange that extends down to fit over said baseplate when installed providing an interference fit, and said plug extends from a surface opposite of said concave surface; or
a base, a glenosphere extending at an angle from said base, and said plug extends from a surface of said base opposite said glenosphere.

75. A modular shoulder prosthesis system comprising:
a baseplate having
a base with a plurality of attachment holes passing therethrough and a pair of attachment points on opposed outer circumferential sides of said base, said attachment points extend at least in from said outer circumferential sides and below a mounting surface of said base, and
a central stem extending from a bottom surface of said base and axially centered with one of said plurality of attachment holes;
a modular component configured to be removably attached to said base, said modular component having a plug for insertion into at least one attachment hole of said base;
a humeral base that is either a humeral stem or a second baseplate, said humeral base having a receiving cavity extending in from one face; and
a modular humeral component configured to cooperate with said modular component, said modular humeral component having a post configured for removable insertion into the receiving cavity of said humeral base, and
wherein said baseplate is capable of attachment to different modular components and said humeral base is capable of attachment to different modular humeral components to facilitate both traditional anatomic total shoulder replacement and reverse total shoulder replacement with a change in the modular component and change in the modular humeral component; and
said attachment points are configured to provide leverage points to facilitate removal of said baseplate from a patient.

76. The system according to claim 75, wherein said baseplate includes a pair of leverage notches on opposed outer circumferential sides of said base, said leverage notches extend down from said mounting surface of said baseplate and in from said outer circumferential sides of said base.

77. The system according to claim 76, wherein
said modular component includes a pair of protrusions extending from opposing sides of said modular component, said protrusions configured to align with said leverage notches when said modular component is attached to said baseplate and/or configured to engage with an interference fit said leverage notches when said modular component is attached to said baseplate; and said protrusions configured to provide second leverage points on which to apply leverage for removal of said modular component after installation onto said baseplate; and/or
wherein said humeral base includes a pair of notches on opposed outer circumferential sides of said humeral base, said pair of notches extend down from a mounting surface and in from said outer circumferential sides of said humeral base.

78. The system according to claim 76, wherein said attachment points are laterally offset from said leverage notches.

79. The system according to claim 75, wherein said modular component includes
a TSR modular component having
a base having a concave surface and a peripheral flange that extends down to fit over outer circumferential sides of said base of said baseplate when installed providing an interference fit, and
said plug extends from a surface opposite of said concave surface; or
a r-TSR modular component having
a base having a peripheral flange that extends down to fit over outer circumferential sides of said base of said baseplate when installed providing an interference fit,
a glenosphere extending from said base, and
said plug extends from a surface of said base opposite said glenosphere.

80. The system according to claim 75, wherein said modular humeral component further includes
a TSR modular humeral component having
a base having
a receiving cavity,
said plug extends from a surface opposite of said receiving cavity; and
a shell configured to be inserted into said receiving cavity, said shell optionally including a bottom edge to engage a top of said receiving cavity; or
a r-TSR modular humeral component having
a base having an inner head having a hemi-spherical dome;
an outer shell having a hemi-spherical cap that fits over said inner head, said hem i-spherical cap is configured to slide relative about said hemi-spherical dome to allow for dual mobility between said hemi-spherical cap and said hemi-spherical dome; and
said stem extending from said base on a surface opposing said inner head.

81. The system according to claim 75, wherein said post of said modular humeral component is eccentrically offset or manually offsetable from an axial center of said modular humeral component.
